(19) United States
(12) Patent Application Publication
(10) Pub. No.: US 2019/0187048 A1
(43) Pub. Date: Jun. 20, 2019

(54) SPECTROSCOPIC SYSTEMS AND METHODS FOR THE IDENTIFICATION AND QUANTIFICATION OF PATHOGENS

(71) Applicant: MONASH UNIVERSITY, Clayton (AU)

(72) Inventors: Bayden Robert Wood, Upwey (AU); Philip Robert Heraud, Upwey (AU); David Perez Guaita, South Yarra (AU); Kamila Natalia Kochan, Caulfield (AU)

(21) Appl. No.: 16/326,686

(22) PCT Filed: Aug. 19, 2017
(86) PCT No.: PCT/IB2017/055028

§ 371 Date: Feb. 19, 2019

(30) Foreign Application Priority Data
Aug. 19, 2016 (AU) 2016903287

(51) Int. Cl. G01N 21/35; C12Q 1/04; G01N 21/552; G01N 21/65

(52) U.S. Cl. CPC G01N 21/35; C12Q 1/04; G16B 5/00; G01N 21/65; G01N 21/552

(57) ABSTRACT

A system for detecting a disease agent in a sample derived from a patient biofluid may comprise a receiver coupled to a communication network and a controller coupled to the receiver. The controller may comprise a processor and a memory. The controller may be configured to generate an infrared spectrum of the sample. The sample spectrum may comprise one or more sample spectral components, the sample spectral components comprising a sample wavenumber and a sample absorbance value. A set of reference spectral models may comprise one or more reference spectral components. The reference spectral components may comprise a reference wavenumber and a reference absorbance value. The reference spectral components may comprise one or more pathogen characteristics associated with sepsis. The one or more sample spectral components may be classified as pathogenic using the reference spectral models. Pathogen data may be generated using the classified sample spectral components.

### CROSS-REFERENCE TO RELATED APPLICATIONS

This application claims priority to Australian Provisional Patent Application No. 2016903287, filed on Aug. 19, 2016, and titled “Spectroscopic Method and Device for Diagnosis of Pathogens causing Sepsis,” the content of which is hereby incorporated by reference in its entirety.

### BACKGROUND

### Sepsis

Sepsis is caused by a patient's immune response triggered by infection by a pathogen. The infection is most commonly bacterial, but it can also be from fungi, viruses or parasites. Sepsis is life-threatening when the patient's response to infection injures its own tissues and organs. Common signs and symptoms include fever, increased heart rate, increased breathing rate, and confusion. But in the very young, old, and people with a weakened immune system, there may be no symptoms of a specific infection and the body temperature may be low or normal rather than high. Disease severity partly determines the outcome with the risk of death from sepsis being as high as 30%, severe sepsis as high as 50%, and septic shock as high as 80%. The total number of cases worldwide is unknown as there is little data from the developing world but it is estimated that sepsis affects millions of people a year.

In the past, diagnosis has been based on assessment of at least two systemic inflammatory response syndrome (SIRS) criteria due to a presumed infection. In 2016 screening by SIRS was replaced with qSOFA (quick Sepsis-related Organ Failure Assessment), which is two of the following three: increased breathing rate, change in level of consciousness, and low blood pressure. But this can be inaccurate, because other conditions such as anaphylaxis, adrenal insufficiency, low blood volume, heart failure, and pulmonary embolism often have signs and symptoms that are very similar to sepsis.

Accordingly, blood culture diagnostic methods are usually undertaken before treatment is started. Conventional blood culture diagnostic methods before identification takes more than one day to diagnosis, often after the fact. New molecular methods such as those described by Dark et al. (Intensive care medicine, 2015, 41, 21-33) include mass spectroscopy and multiplex real-time PCR (Septifast) and can yield results in 6 hours from blood sampling and require complex amplification steps.

The current techniques for sepsis diagnosis are not practical for point-of-care testing. This means that medical treatment, which needs to be applied immediately because of the critical nature of the problem is often delayed, not specific to the type of pathogen, and based on subjective diagnosis.

Other evolving technologies like Integrated Comprehensive Droplet Digital Detection integrates real time detection with DNAzyme-based sensors, droplet microencapsulation and a high throughput 3D particle counter system to detect from 1-10,000 bacteria per ml of blood (Kang et al., Nature Communications, 2014, 5, 5427). While this approach has potentially very high sensitivity it has only been proven in spiked blood samples and requires skilled personnel and very expensive equipment.

T2 Magnetic resonance has been recently applied to detect fungal infection from Candida to a detection limit of one colony-forming unit in a blood sample but the test still takes about 3 hours (Neely et al., Science Translational Medicine, 2013, 5, 182ral154). All of these techniques rely on the differences in the DNA sequences between the host and pathogen and require some type of DNA extraction or amplification process.

Accordingly, there is a need for an efficient point-of-care test for assessment of emergency febrile patients for sepsis to provide specific and more timely treatment.

### Vibrational Spectroscopy Analysis of Blood

Techniques such as ATR-FTIR have been utilized for diagnosis of blood infections. For example, KHOSHMANESH, A. et al. (Analytical Chem. 86, pp 4379-4386) quantified parasites in fixed red blood cells using the changes induced in the lipids of the RBCs by the malaria parasite using ATR-FTIR spectroscopy.

Sitole et al (OMICS: A Journal of Integrative Biology, 18 (8) pp. 513-523) identified differences between the ATR-FTIR spectra if controls and HIV infected samples, located in bands of lipids, proteins and fatty acids.

Chunder (U.S. Pat. No. 8,822,928) has presented the use of IR for diagnostic pathologies in inner organs using the width and absorbance of different IR bands of the spectra.

El-Sayed et al. (U.S. Pat. No. 6,379,920) discloses the detection of bacterial infection in blood samples by acquiring an infrared, Raman or fluorescence spectrum. In particular, El-Sayed teaches a method for diagnosing bacteria in a biologic sample by analysing a sample of infected serum. Using a spectrometer, the spectrum of a previously obtained reference serum is subtracted from the spectra of the infected serum, and the resulting differential spectra are compared with reference spectra of bacteria in saline to determine the specific bacteria present in the infected serum. However, this approach has the drawback that it does not take into account the metabolic variability between sera caused by a number of factors including nutrition.

Wood et al. (International Patent Application No. PCT/AU2015/000631) discloses a method for detecting infectious blood borne diseases using multivariate analysis of the ATR-FTIR spectrum. In particular, Wood teaches a method for detecting disease agents such as malaria parasites in blood and HIV, HBV, HCV infections in serum, plasma and whole blood.

However, all the aforementioned studies focus on the detection of a broad collection of signatures in the spectrum and not a unique signature directly associated with the presence of a pathogen species. In particular, the aforementioned conventional techniques rely on the disease causing changes to the serum metabolites but do not directly detect the pathogen causing sepsis. Serum is a complex matrix with thousands of metabolites, and the detection of one or more unique signatures is more highly affected by interference caused by the complex metabolism of the patient. The above aforementioned techniques are unable to quantify a pathogenic load of serum.

There is therefore a need to develop point-of-care vibrational spectroscopy based approaches, which directly and robustly detect spectroscopic bands associated with the pathogen.

### BRIEF SUMMARY

In one form, the technologies discussed herein relate to the field of spectroscopic detection of pathogens found in liquids including blood, serum, water, saline, milk, urine, saliva and other body fluids. The pathogens detected may include bacteria, viruses, prions and fungi, as well as pathogens associated with sepsis.

The systems and methods herein may include direct spectroscopic identification of a pathogen. This may include a preparation phase using a biological sample for spectroscopic analysis, and may include filtration, purification, concentration, deposition, and/or drying of the patient sample or pathogen followed by direct spectroscopic identification based on a unique molecular phenotype without the need for culturing.

In some variations, point-of-care taxonomic identification of pathogens may include the steps of identifying the presence or absence of a list of pathogens that are associated with sepsis, based on their infrared spectrum derived from clinical blood samples matched to a reference spectral database. For example, the list of pathogens may comprise a predetermined or pre-selected list of the twelve (or other number) of the most common or likely pathogens that cause sepsis. For example, the pathogen list may be include, but is not limited to, Klebsiella pneumonia, Escherichia coli, Pseudomonas aeruginosa, Staphylococcus epidermidis, Streptococcus dysgalactiae, Staphylococcus capitus, Enterococcus faecalis, Staphylococcus aureus, Hafnia alvaris, Stenotrophomonas maltophila, Enterococcus faecium, and Candida parapsilosis.

In some variations, indicia of sepsis may be aggregated by using particles such as silica particles to trap species such as bacteria, viruses and fungi. These species may then be detected using a spectroscopic device. In some variations, ATR-IR, ATR-FTIR or Raman spectroscopy may be used detect various sample characteristics, including but not limited to various vibration, absorption and/or scattering characteristics of the samples.

In some variations, spectrographic data obtained during the detection phase may be analysed to detect the presence and/or other characteristics of one or more pathogens in the sample. The processing may include the use of linear multivariate and/or non-linear support vector machine/neural network modeling approaches for pathogen identification and quantification.

It will be convenient to hereinafter describe the variations of the invention in relation to diagnosis of sepsis using blood serum, however, it should be appreciated that the variations described may be applied to other biofluids for the detection and ultimate identification of pathogens, and/or other clinical conditions or diagnoses

Where used herein the term “biofluid” is intended to include whole blood, blood derivatives or constituents such as serum or plasma or other fluids created or stored by body tissue including cerebrospinal fluid, urine, water, milk and saliva including combinations thereof.

In one aspect, the technology provides a method of screening for a pathogen in a patient, comprising extracting the pathogens from a patient biofluid sample, filtering the biofluid sample to remove at least a portion of the liquid component and collect the larger particle components, delivering an electromagnetic beam from the infrared-visible spectrum through the filtered biofluid sample, and detecting the presence or absence of the pathogen(s).

The extraction of pathogens from the patient biofluid may include isolating certain particulate matter from the biofluid or otherwise removing at least a portion of the liquid from the sample using a filter, suspending the filtered sample in a solvent such as ultrapure water or another solvent, and concentrating the pathogen in an amount of solvent, such as water to form a concentrated sample. The amount of the solvent may be pre-determined based on the spectroscopy machine or based on the original biofluid sample volume. The concentrated sample may be applied to an ATR crystal or infrared/Raman substrate and subjected to a beam of infrared or visible wavelength electromagnetic energy, such as an evanescent infrared beam.

In a second aspect, a method of screening for a pathogen in a patient may comprise centrifuging a biofluid sample from the patient in the presence of particles, delivering an electromagnetic beam from the infrared-visible spectrum through the patient biofluid sample, and detecting the presence of particles associated with at least one pathogen. The centrifugation may include, for example, the use of a serum separator tube (SST).

One or more pathogens may be identified by detecting a molecular phenotype specific to the pathogen by comparison of absorption or scattering of the electromagnetic beam by the sample with a reference database and/or model of the pathogen.

Additionally, or alternatively, the concentration of pathogens in the sample and pathogenic load in a patient may be quantified by comparing absorption of the electromagnetic beam by the biofluid sample with calibration models (e.g., reference data set) to quantify the number of pathogen cells present in the biofluid sample.

In a third aspect of variations, a method of detecting a pathogen in a patient may comprise the steps of delivering an electromagnetic beam from the infrared-visible spectrum, through a substrate in contact with a sample derived from a patient biofluid to create an infrared sample spectrum representative of the biofluid. The sample spectrum may comprise one or more spectral components with each component including a wavenumber and absorbance value. Analysis of the absorption of an electromagnetic beam by the sample may be performed to detect the presence of molecular differences between pathogens by providing a reference database of spectra for each pathogen that can be used to derive models that have one or more database spectral components of a wavenumber and an absorbance/intensity value. The database spectral components may be used to identify disease agents as specified in Table 1 for infrared spectroscopy and Table 2 for Raman spectroscopy. The reference database may be used to classify whether has one or more database spectral components correspond to one or more spectral components that correspond to the pathogen. A list of corresponding database components may be identified and generated. The total absorbance of the ATR spectrum may be generated based on the integrated area of either one or more spectral windows. Alternatively, the integrated area of the entire spectrum 4000-700 cm⁻¹ may be used to quantify the pathogen load based on calibration models developed for each pathogen.

The method may further comprise quantifying the pathogen by comparing absorption of the electromagnetic beam with calibration models to quantify the number of pathogen cells present in the sample. This may be achieved by measuring the integrated absorbance/intensity of one or several spectral windows or by integrating the area under the entire spectrum. The method may also include identifying the molecular phenotype, which is specific for a particular type of pathogen by comparison of absorption of the infrared beam by the sample with a reference database or model for the pathogen phenotype.

### Systems & Devices

Preferably the methods of the present technology are carried out in conjunction with an automated system or device.

In a fourth aspect of variations, a computer readable storage medium for storing in non-transient form an application for executing a method of detecting a pathogen associated with sepsis in a sample derived from a patient biofluid, comprising recording an infrared spectrum representative of the sample, comparing the spectrum to a reference database of spectral models to identify one or more spectral components of wavenumber and absorbance/intensity of the sample. The spectral components identify pathogens. A list of sample components may be identified and compiled corresponding to a respective pathogen spectrum in the database. The recording, comparing and/or compiling functions may be automated.

The computer readable storage medium may further include comparing the spectrum to a reference database of calibration models to identify one or more spectral components of wavenumber and absorbance/intensity of the sample. The spectral components quantify the number of pathogenic cells present in the sample.

A fifth aspect of the variations provides a system configured to detect a disease agent in a sample derived from a patient biofluid, the system comprises a memory and a controller with a processor and a predetermined instruction set to record a sample infrared/Raman spectrum representative of the sample. The sample spectrum having one or more spectral components, each component having a wavenumber and absorbance value. A reference database of spectral models may be provided. Each model may have one or more database spectral components of a wavenumber and an absorbance value. The database spectral components may identify pathogens associated with sepsis. A determination may be made whether the reference database has one or more database spectral components corresponding to one or more sample spectral components, and a list of corresponding database components may be identified and compiled.

In a sixth aspect of variations, an application may be adapted to enable the detection of a disease agent in a sample derived from a patient biofluid, said application comprising a predetermined instruction set adapted to enable a method comprising the steps of: recording a sample infrared spectrum representative of the sample, the sample spectrum having one or more spectral components, each component having a wavenumber and absorbance value; providing a reference database of spectral models, each model having one or more database spectral components of a wavenumber and an absorbance value, wherein the database spectral components quantify the number of pathogenic cells; determining whether the reference database has one or more database spectral components corresponding to one or more sample spectral components; and compiling a list of corresponding database components identified and their quantification.

In some variations, a computer readable storage medium may be provided for storing in non-transient form, an application for executing a method of detecting a pathogen associated with sepsis in a blood sample is provided, comprising: recording an IR and/or Raman spectrum after pathogen preconcentration and isolation. Treated or untreated biofluid, particularly from a SST tube using particles; pre-processing the spectrum to enable comparison to a model spectrum in a data base; and transferring the spectrum to a remote central database to correlate the spectrum with a pathogen database, which may be updated in real time or periodically with epidemiological data. The recording, pre-processing and/or transferring functions may be automated. For example, one of more of these functions are performed without requiring user input.

It will be clear to the person skilled in the art that in addition to the application being stored on a computer readable storage medium, it may be stored in the cloud or other computing equivalent. There is thus provided an application adapted to enable the detection of a disease agent in a blood sample, the application comprising a predetermined instruction set adapted to enable a method comprising: creating a sample infrared and or Raman spectrum representative of the pathogen spectrum having one or more spectral components, each component having a wavenumber and absorbance value; providing a reference database of spectral models, each model having one or more database spectral components of a wavenumber and an absorbance value, wherein the database spectral components identify disease agents; determining whether the reference database has one or more database spectral components corresponding to one or more sample spectral components; and compiling a list of corresponding database components identified.

Thus, some methods involve generating an IR and/or Raman spectrum of a biofluid sample using a standard FT-IR spectrometer and a diamond crystal ATR accessory or a Raman spectrometer/microscope and the model function to derive a diagnosis. Thus, the method may be performed using equipment that is relatively small size that is suitable for field use, even in remote locations.

In some variations, a system may be provided for directly detecting a pathogen in a serum, blood or bio-fluid sample. The system may comprise a spectrometer to capture an IR and/or Raman spectrum, and a computer. The spectrometer may generate an IR or Raman spectrum representative of the pathogen by isolating it from the serum, blood or bio-fluid sample. The spectrum may be pre-processed by smoothing, baseline corrected by taking a first or second derivative or applying or a linear/polynomial base line correction, and normalizing using standard normal variate (SNV) or another normalisation. The computer may apply the pre-processed spectrum to a reference database of spectral models to identify one or more spectral components of wavenumber and absorbance representative of the molecular phenotype of the pathogen within the serum, blood or bio-fluid sample. The spectral components for each of the pathogens may be explicitly identified. The computer may compile a list of sample components identified corresponding to a respective spectral model of the database.

Other aspects and preferred forms are disclosed in the specification and/or defined in the appended claims, forming a part of the description of the technology.

Advantages provided by the method of the present technology may include providing a point-of-care test for assessment of emergency febrile patients for sepsis to enable specific and/or timely treatment. The systems, device, and methods disclosed herein do not require culturing of the microbes and is faster than conventional sepsis detection methods. The methods do not require any reagents and do not require significant training and can be taught to a health worker in as little as 20 minutes or so. The methods may further be highly sensitive and have high specificity for discriminating between sera samples containing pathogens and those that do not contain pathogens. In some variations, the methods may also be used to discriminate between different pathogens including gram positive and gram negative bacteria and fungi associated with sepsis along with a set of most common bacteria associated with sepsis.

Further scope of applicability of variations of the present technology will become apparent from the detailed description given hereinafter. However, it should be understood that the detailed description and specific examples, while indicating preferred variations of the technology, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other variations of the present application may be better understood by those skilled in the relevant art by reference to the following description of variations taken in conjunction with the accompanying drawings, which are given by way of illustration only, and thus are not limitative of the disclosure herein, and in which:

FIG. 1 illustrates an overview of a protocol for extraction and preconcentration of pathogen, followed by vibrational-spectroscopy based detection, according to the method of the present technology.

FIG. 2 illustrates exemplary plating results obtained for spiked serum 210 (top row) and solution obtained after filtration and re-suspension 220 (bottom row). After spiking (as well as filtration and re-suspension) the suspensions were diluted in order to obtain HBA plates possible to count.

FIG. 3 illustrates the detection of a pathogen isolated and preconcentrated on the surface of the ATR crystal by showing the several spectra of acquired on the empty crystal (blanks) and the spectra of the crystal loaded with 1.71×10⁴ (302) and 1.71×10⁵ (304) colony forming units of Staphylococcus aureus.

FIG. 4 illustrates ATR-FTIR spectra of twelve common bloodstream pathogens.

FIG. 5 illustrates the Raman spectra of ten common pathogens.

FIG. 6 depicts, for each sample tested, the predicted probability of being classified as the three different pathogens classes included in the SIMCA multi-class classification.

FIG. 7 shows the quantitative relationship between the area of the amide bands and the amount of bacteria on the surface of the ATR crystal.

FIG. 8 illustrates a method of developing data models and a cloud based diagnostic platform for the identification of pathogens that cause sepsis.

FIG. 9A illustrates ATR-FTIR spectra of pure particles obtained from SST tubes. FIG. 9B illustrates Raman spectra of pure particles obtained from SST tubes.

FIG. 10 is a block diagram of a pathogen identification system for that may include detection, quantification and identification of a pathogen from an Infrared and Raman Spectrum.

FIG. 11 depicts a block diagram of another variation of an identification and quantification system.

FIG. 12 depicts a flowchart of a quality control method for identifying and quantifying a pathogen in a serum sample.

FIG. 13 depicts a flowchart of a quality control threshold test to determine if the spectrum has sufficient absorbance or intensity for modeling.

FIG. 14 depicts a flowchart of steps performed by a classifier developer.

FIG. 15 depicts a flowchart of a method for identifying and quantifying a pathogen in a serum sample.

FIG. 16 schematically depicts an exemplary system architecture of a cloud-based spectroscopy system.

### DETAILED DESCRIPTION

### Direct Detection and Identification of Pathogen

In some variations, a pathogen in a sample may be detected and identified based on its IR/Raman spectral profile, which is representative of its specific molecular phenotype. The pathogens may be isolated through filtration and then preconcentrated from the biofluid onto the surface of an ATR crystal or substrate for infrared and/or Raman spectroscopy.

The method may include the steps of collecting whole blood into serum separation tube, and generating a serum such as through centrifugation (e.g., 100-10000 RCF, 1-20 minutes). The serum may be filtered using a filter system with pore sizes selectable in a range between about 0.015 micron to 1 micron pore size that permits the filter system to trap any pathogens of a serum sample on the surface of a filter. Filter materials may be chosen depending on the need for hydrophobic or hydrophilic filter characteristics. The filter may be washed using, for example, ultrapure water or another solvent including alcohol, xylene, acetone and other inorganic or organic solvents. Washing may remove serum residuals in the filter along with platelets. Existing pathogens trapped on the filter surface may be resuspended using a small amount of ultrapure water or any type of solvent. The resuspended solution may be pre-concentrated using a centrifuge device having a filter as described herein. The pre-concentrated sample may be dried directly on the surface of the ATR crystal or a reflective Raman slide or other infrared/Raman spectroscopic transmitting or reflective substrates. For example, drying may be performed using a dried gas or air stream. The substrates may be placed in an oven, drying cabinet or use a dehydrating solvent to resuspend the pathogens in alcohol instead of ultrapure water. IR light or an IR/visible/UV laser beam may be delivered to a biofluid. The sample spectrum may comprise one or more spectral components, each component having a wavenumber and absorbance values in case of the IR. Where Raman spectroscopy is used, the spectral components may comprise a Raman intensity and Raman wavenumber shifts.

One or more pathogens may be identified and/or quantified according to its IR or Raman spectra using models constructed from a reference spectral database of pathogens measured under similar experimental conditions. Identification and/or quantification may be performed by providing a reference database of spectral models with each model having one or more database spectral components of a wavenumber and absorbance values. The database of spectral components may be used to identify and quantify disease agents. The reference database may be used to determine whether one or more database spectral components corresponds to one or more sample spectral components. In other words, one or more sample spectral components may be classified as pathogenic using the reference spectral models. A list of corresponding database components may be identified and compiled. For example, pathogen data may be generated using the classified sample spectral components.

Pre-concentrating and isolating the pathogens from the biofluid may be carried out using any process that separates the pathogen from the biofluid and other components (e.g., cells, proteins, metabolites) and pre-concentrates the pathogenic suspension to a minimum volume of ultrapure water or other solvent. These steps may include filtration (e.g., using a 0.015-1 micron filter of either hydrophobic or hydrophilic characteristics) depending on the type of pathogen. Magnetic ionic liquids may also be used as a filter.

In some variations, a multivariate model may be used to determine the presence of a pathogen by evaluating the absorbance of the pathogen and comparing the absorbance of the sample spectrum with the detection threshold of the spectroscopic device. If the signal meets a predetermined signal quality threshold, then the spectrum may be compared to a reference database to identify and quantify the pathogen.

In some variations, the method may further include selecting one or more spectral subsets or windows when comparing the spectral data to the reference database, which may improve processing efficiency or speed.

Differences in the composition of one or more pathogens (species or even strains) may be determined using ATR and/or Raman spectroscopy. For example, amide I (1650 cm⁻¹) and amide II bands (˜1540 cm⁻¹) along with lipid bands (3100-2800 cm⁻¹) and the ester carbonyl (1740 cm⁻¹) may be used to distinguish between Gram positive and Gram negative bacteria. In addition, bands at 1102 cm⁻¹ (peptidoglycan), around 1218 cm⁻¹ (teichoic acid), and 1248 cm⁻¹ (amide III, peptidoglycan), are associated with Gram positive bacteria and can be used to distinguish Gram-positive and Gram-negative bacteria. In particular, fungi such as Candida have a series of intense bands from C—O stretching modes from carbohydrate moieties in the range of 1200 cm⁻¹ to 1000 cm⁻¹. DNA from pathogenic organisms present in the serum may be associated with absorption of specific wavelengths of IR energy at about 1050 cm⁻¹, 1080 cm⁻¹, 1225 cm⁻¹, and 1705-1720 cm⁻¹. These wavelengths may be used to determine the presence of pathogens corresponding to sepsis in a patient.

The ratios and the IR/Raman spectral profile corresponding to a pathogen may be very specific so as to allow matching to a reference spectral database or a multivariate model. These methods may allow a system to distinguish gram positive and gram negative from fungi, as well as allow identification of the twelve most common pathogens or any others associated with sepsis.

### Spectroscopy-Based Analysis

Infrared (IR) and Raman spectroscopy relates primarily to the absorption (IR) or scattering (Raman) of light by molecular vibrations having wavelengths in the infrared segment of the electromagnetic spectrum, that is energy of wavenumber between 200 and 4000 cm⁻¹. The structure of almost all biological molecules includes moieties that absorb energy in the IR segment or scatter energy from the VIS segment of the electromagnetic spectrum. Thus, an IR spectrum and Raman of a clinical sample is representative of its main biological components and can be in the nature of a ‘metabolic fingerprint’.

ATR is a sampling technique that may be used in conjunction with IR. The sample may be put in contact with the surface of a crystal having a higher refractive index than the sample. A beam of IR light may be passed through the ATR crystal in such a way that it reflects at least once off of the internal surface in contact with the sample. This reflection forms an evanescent wave which extends into the sample. The penetration depth into the sample depends on the wavelength of light, the angle of incidence, and the indices of refraction for the ATR crystal and the medium being probed. The number of reflections may be varied. The beam is then received by a detector as it exits the crystal.

Table 1 shown below lists typical IR bands of biological compounds and their respective assignments. Pathogens may have various ratios and combinations of these bands that act as unique molecular phenotypes. The pre-processed spectrum may be matched to a database using a least squares fitting procedure. A calculated value above r²>0.9 (where a 1.0 is a perfect match) may be set as a predetermined threshold corresponding to identification of a specific pathogen. A calculated value of r² between about 0.5 and about 0.9 may correspond to presence of one or more pathogens.

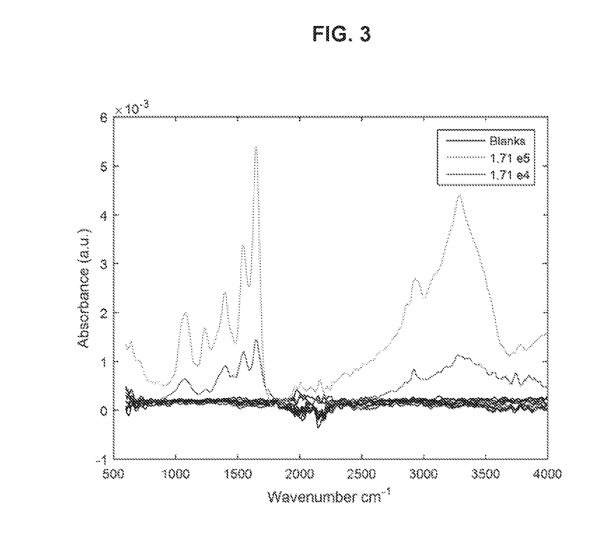

TABLE 1

| Wavenumber | | | Referenced to |
|---|---|---|---|
| (cm⁻¹) | Assignment | Analytes | FIG. 3 |
| 930-1300 | vₛ (C—O) | Saccharides | 22 |
| 1000-1150 | vₛ (P—O) | Phospholipids, DNA | 22 |
| 1150-1300 | vₐₛ (P—O) | Phospholipids, DNA | 22 |
| 1200-1400 | Amide III | Proteins | 21 |
| 1430-1480 | δ(CH₃), δ(CH₂) | Lipids | 20 |
| 1480-1600 | Amide II | Proteins | 19 |
| 1600-1720 | Amide I | Proteins | 18 |
| 1700-1760 | vₛ (C═O) | Lipids | 17 |
| 2840-2860 | vₛ (CH₂) | Lipids | 16 |
| 2860-2870 | vₛ (CH₃) | Lipids | 16 |
| 2870-2950 | vₐₛ (CH₂) | Lipids | 16 |
| 2950-2990 | vₐₛ (CH₃) | Lipids | 16 |
| 3000-3020 | v (CH) | Lipids | 16 |
| 4000-4550 | v (CH) combinations | Lipids | |
| 4550-5000 | v (NH) v (OH) combinations | Proteins, saccharides | |
| 5600-6050 | 1° overtone v (CH) | Lipids | |
| 6700-7150 | 1° overtone v (NH) v (OH) | Proteins, saccharides | |
| 8000-9100 | 2° overtone v (CH) | Lipids | |
| 9100-10500 | 2° overtone v (NH) v (OH) | Proteins, saccharides | |
| 11520-11760 | 3° overtone v (CH) | Lipids | |
| 11765-12900 | 3° overtone v (NH) | Proteins, saccharides | |

Table 2 lists observed wavenumber values (cm⁻1) for the most prominent Raman bands of pathogenic bloodstream bacteria and fungi with assignments obtained using a 532 nm laser line. The following abbreviations are used in Table 2: ^{a}v—stretching; def—deformation; br—breathing; sym—symmetric; asym—asymmetric; Phe—phenylalanine; Trp—tryptophan; Tyr—tyrosine; T—thymine; A—adenine; G—Guanine; C—cytosine; U—uracil.

| Wavenumber/cm⁻¹ | Band Assignment | Compound |
|---|---|---|
| 3045 | v(CH) | Proteins |
| 2934 | aromatic and aliphatic | Proteins |
| | v(CH) | |
| 2874 | aliphatic v(CH) | Proteins |
| 2854 | v_{sym} (CH₃) | Lipids |
| 2722 | aliphatic v(CH₂, CH₃) | lipids/proteins |
| 1745 | v(C═O) ester | Lipids |
| 1673 | Amide I | Proteins |
| 1655-1680 | | |
| 1657 | v(C═C) | Lipids |
| 1619 | v(C═C), Tyr, Trp | Proteins |
| 1602 | mitochondrial activity/v(C═C), Phe, Tyr | Ca²⁺ influence/proteins |
| 1584 | G, A/Phe | nucleic acids/proteins |
| 1574 | | |
| 1554 | | |
| 1483 | G, A/CH def | nucleic acids/proteins |
| 1449 | C—H₂ def/CH def | proteins/lipids |
| 1336 | A, G/C—H def | nucleic acids/proteins |
| 1320 | G/C—H def | nucleic acids/proteins |
| 1303 | Amide III/CH₂ twist | proteins/lipids |
| 1262 | T, A/C—H bend, Amide III/═CH bend | nucleic acids/proteins/lipids |
| 1244 | Amide III | Proteins |
| 1212 | T, A/Amide III | nucleic acids/proteins |
| 1184 | Tyr, Phe, C—H bend | Proteins |
| 1157 | v(C—C, C—N) | Proteins |
| 1130 | v(C—N) | Proteins |
| 1091 | v_{sym}(O—P—O)/v(C—N) | nucleic acids/proteins |
| 1079 | chain v(C—C) gauche | Lipids |
| 1063 | chain v(C—C) trans | Lipids |
| 1004 | sym. ring br. Phe | Proteins |
| 857 | Tyr. ring br./v_{sym}(C—C—N⁺) | proteins/lipids |
| 814 | v_{asym}(O—P—O)/ring br. Tyr | RNA /proteins |
| 784 | v(O—P—O)/U, T, C | DNA/nucleic acids |
| 760 | T/ring br. Trp | nucleic acids/proteins |
| 723 | A | nucleic acids |

DNA from pathogenic organisms in the serum may correspond with absorption of specific wavelengths of IR energy at about 1050 cm⁻¹, 1080 cm⁻¹, 1225 cm⁻¹, and between 1705 cm⁻¹ and 1720 cm⁻¹, but other bands from lipids, proteins and carbohydrates are also to distinguish pathogens (as described in Table 1). Accordingly, absorption may be used to determine the presence of pathogens causing sepsis in a patient. Nucleic acids from lysed cells from the patient pass through the filter.

### Direct Detection of Pathogens Using a Spectroscopic Device

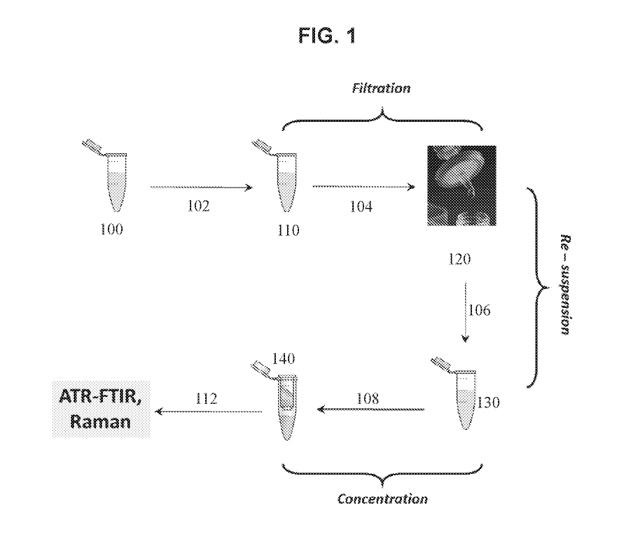

The systems, devices, and methods described herein may rapidly identify one or more pathogens in serum. This may aid targeted treatment that may improve patient outcomes. FIG. 1 illustrates an overview of a protocol for extraction and preconcentration of pathogen, followed by vibrational-spectroscopy based detection. A blood sample 100 is centrifuged 102 to obtain serum 110. Serum 110 is subsequently filtered 104 resulting in pathogens being trapped on the filter 120. A range of filter sizes may be employed in the range from about 0.015 to about 1 micron depending on the target pathogen. Viruses would employ filter sizes at the lower end of this range. Bacteria would employ filters with pore sizes less than 0.22 microns, while filters with pore sizes above 0.22 microns may be suitable from bloodstream parasites, for example. Pre-filtration with larger pore sizes may facilitate the subsequent extraction of pathogens using filters with smaller pore sizes, e.g. viruses. Filter material can vary depending on the fluid being filtered. Biofluids subjected to solvent extracts may employ inorganic filters such as, but not restricted to, those composed of aluminium oxide. Filter materials may also have hydrophilic or hydrophobic properties. Hydrophilic filter materials such as, but not restricted to, cellulose acetate, polyether sulfone, polytetrafluoroethylene, may be best suited to body fluids containing proteins such as serum, plasma, cerebrospinal fluid etc. as proteins bind less readily to these materials. Hydrophobic filter materials such as, but not restricted to, polycarbonate or polyethylene, are suitable for organic extracts of body fluids. Hydrophobic filter materials may be rendered more hydrophilic and thus better suited to filter body fluids directly by applying wetting agents such as polysorbates (palmitate esters of sorbitol and its anhydrides copolymerized with ethylene oxide). Once the body fluid or its solvent extract has been filtered, the filter 120 is then washed and pathogens are re-suspended in ultrapure water, or organic or inorganic solvent 106, resulting in a water or solvent suspension of pathogen 130. The suspension 130 is subsequently concentrated 108 in a microcentrifuge device 140 and the obtained sample is subjected to Raman (dried on a slide made of appropriate material, e.g. CaF₂) and/or ATR-IR measurement 112 (dried directly on the crystal). The drying process may be assisted by a stream of air or gas or a capillary wicking system.

In some variations, vibrational-spectroscopy based identification and/or quantification of pathogens from serum samples may comprise the steps of extracting a pathogen and pre-concentration from serum sample. Deposition and drying of the purified pathogen extract may be performed on an ATR crystal or Raman reflective substrate. Classification and quantification of the extracted pathogen may be performed using vibrational spectroscopy. For example, a ‘dead-end’ approach may be used to extract one or more pathogens from a filtered serum sample. A filter system may be used to extract a pathogen through filtration using a filter having a pore size between about 0.015 μm to about 1 μm. The pore size prevents pathogens from going through the filter, because the diameter of pore is smaller than the size of pathogens. Filtration may be followed by washing the residuals out of the serum present in the filter with ultrapure water or organic or inorganic solvents. Pathogens on the filter may be re-suspended using a small amount (e.g. 300 μL) of ultrapure water or the other solvent. The obtained solution of pathogens in water or solvent may be subsequently concentrated using a microcentrifuge device. A microcentrifuge device may include a filter and be configured to concentrate the suspension to about 10 μL-30 μL.

As a result of the extraction, about 10 μL-30 μL of suspension containing the pathogens may be extracted from the serum sample. The extraction procedure may be performed using any amount of serum, but may include a range of between about 1 μL and 20 ml. Larger volumes may be required with samples where pathogen levels are suspected to be at lower levels (e.g., cerebrospinal fluid). The efficiency of the extraction process may be close to 100%.

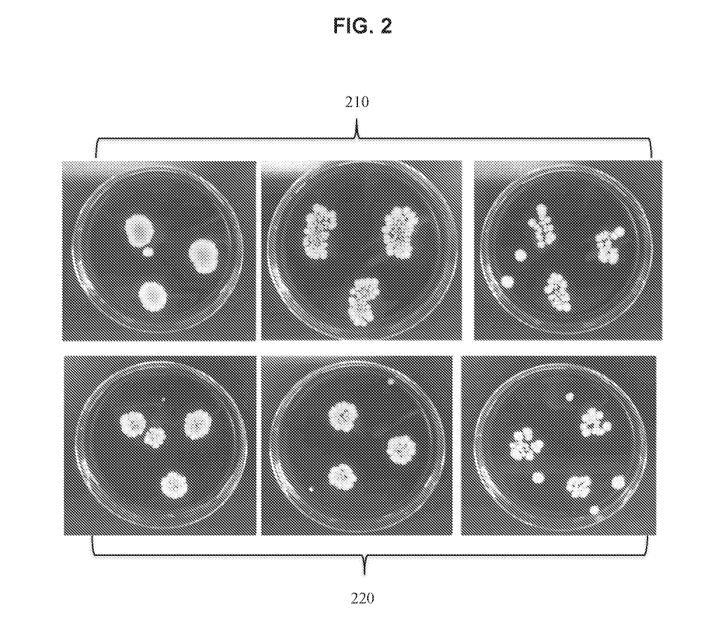

FIG. 2 illustrates results of establishment of concentration of S. aureus in a spiked serum sample and in a solution obtained after filtration and re-suspension of the bacterium. The processed sample obtained through the extraction procedure solution may be subsequently measured with the use of ATR-FTIR technique. In some variations, 0.5 μL of solution may be dried directly on the ATR crystal or other infrared transmitting or absorbing material. Alternatively, the sample may be placed on appropriate material (e.g., Raman grade CaF₂ slide) and a Raman spectrum of the sample may be collected. The methods described herein may use either ‘wet’ or ‘dry’ samples of solution of re-suspended and concentrated pathogens.

The presence of one or more pathogens may be determined using the IR and/or Raman spectra for the solution of bacteria. The IR or Raman signal of a sample may correspond to absence and/or presence of one or more pathogens. FIG. 3 is a plot of spectra recorded after drying two solutions of Escherichia coil, depositing 1.71 e4 and 1.71 e5 compared to a spectrum of the crystal without the bacteria (e.g., blanks). The absorbance is directly proportional to the concentration. 1.71×10⁴ E. coli produce an approximate absorbance of 2×10⁻³ absorbance units, while 1.71×10³ E. coli produce an absorbance of 2×10⁻³ absorbance units. The presence of a pathogen may be determined based on the difference between the pathogen signals and the baseline obtained from the clean crystal or after depositing the pre-concentrated extract (obtained under the same conditions) as the control samples. The detection threshold is dependent on the size of the pathogen, thus Candida sp. has a lower limit of detection compared to E. coli and other bacteria. The generated R and/or Raman spectrum may correspond to one or more characteristics of specific pathogens. Pathogens may be identified by identifying these characteristics in the IR and/or Raman spectrum.

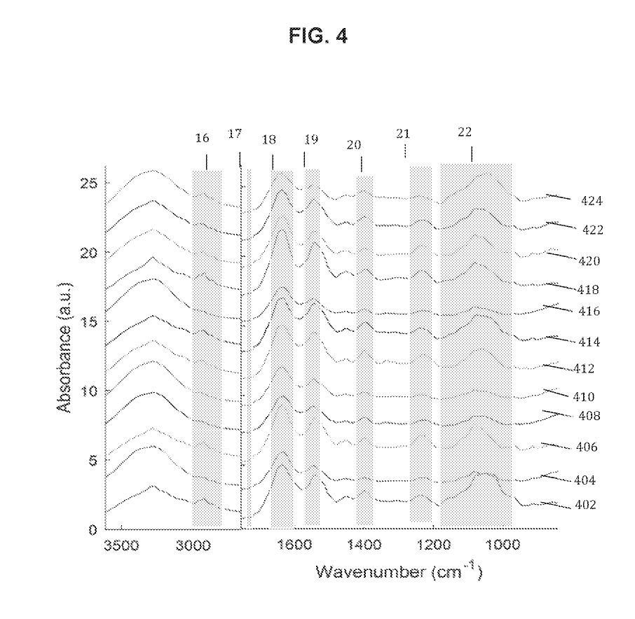

FIG. 4 illustrates ATR-FTIR spectra of twelve common pathogens: Klebsiella pneumoniae 402 (Gram−), Escherichia coli 404 (Gram−), Pseudomonas aeruginosa 406 (Gram−), Staphylococcus epidermidis 408 (Gram+), Streptococcus dysgalactiae 410 (Gram+), Staphylococcus capitus 412 (Gram+), Enterococcus faecalis 414 (Gram+), 416 Staphylococcus aureus (Gram+), Hafnia alvei 418 (Gram−), Stenotrophomonas maltophila 420 (Gram−), Enterococcus faecium 422 (Gram+), and Candida parapsilosis 424 (Yeast) representing the group of yeast, Gram positive and Gram negative bacteria.

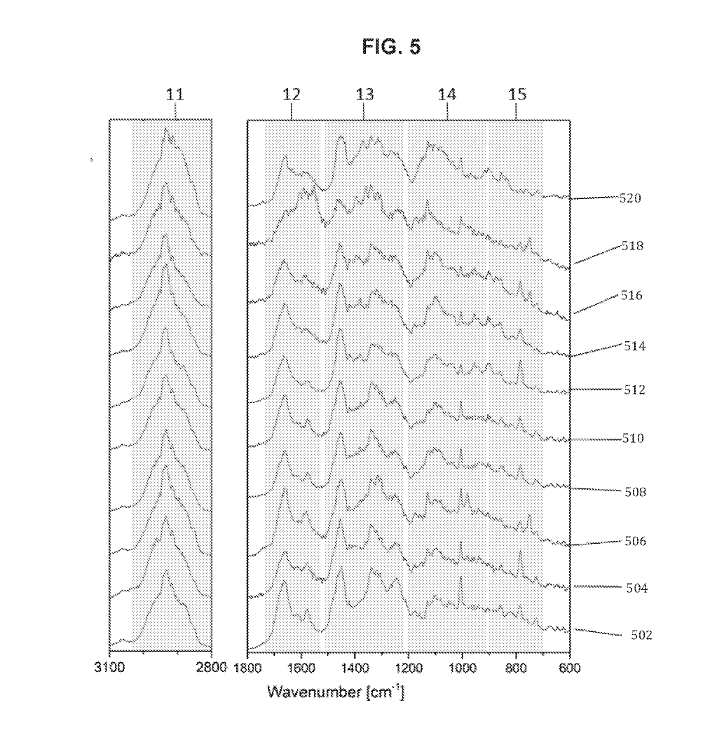

FIG. 5 illustrates Raman spectra of ten common pathogens. FIG. 5 shows a plurality of spectral windows (regions) including a 3100-2800 cm⁻¹ window (11); 1750-1500 cm⁻¹ window (12); 1500-1200 cm⁻¹ window (13); 1200-900 cm⁻¹ window (14); and 900-700 cm⁻¹ window (15). One or more of these regions may be used in the model. For example, the entire spectral region spanning between 3100-2800 cm⁻¹ and 1750-700 cm⁻¹ may provide the best model with sensitivities and specificities approaching 100%. The spectral range between 2800-1750 cm⁻¹ contains no bands of biological origins and bands from carbon dioxide and the diamond ATR crystal, where employed, which could make pathogen identification more difficult. The spectral range above 3100 cm⁻¹ may be dominated by a broad band from the O—H stretching mode of bound water in the sample and may make pathogen identification difficult. The region below 700 cm⁻¹ may be a range where many detectors in IR spectrometers become insensitive with a consequential loss of measurement signal to noise ratio.

Table 3 shown below includes species of pathogen and the spectral windows for distinguishing gram positive from gram-negative bacteria along with spectral windows for unique pathogen identification using multivariate methods. The spectral windows include: spectral window 16 CH stretching region mainly from lipids and proteins (3100-2800 cm⁻¹); spectral window 17 the ester carbonyl region associated with lipids (1750-1700 cm⁻¹); spectral window 18 amide I region (1700-1600 cm⁻¹); spectral window 19 amide H region (1600-1500 cm⁻¹); spectral window 20 carboxylate region from mainly proteins and lipids (1450-1400 cm⁻¹); spectral window 21 asymmetric phosphodiester stretch DNA/amide III region (1300-1200 cm⁻¹); spectral window 22 DNA symmetric stretch and carbohydrate region (1200-800 cm⁻¹).

In some variations, spectral windows 21 and 22 may be used to differentiate gram+ and gram− bacteria, where differences in cell wall components between the two groups are expressed as different bands at 1102 cm⁻¹ (peptidoglycan), around 1218 cm⁻¹ (teichoic acid), and 1248 cm⁻¹ (amide III, peptidoglycan) associated with gram positive bacteria. The numbers next to the species name in Table 3 correspond to numbered spectra in FIG. 4. The spectral windows decrease in relative importance from left-to-right in Table 3. It should be noted that combinations of two or more windows may improve pathogen identification. Spectral window numbers are correlated with the shaded areas in FIG. 4. Gram-negative species of bacteria may be distinguished from Gram-positive bacteria based on the stronger CH₂ and CH₃ stretching vibrations in the 3100-2800 cm⁻¹ region, which correlates to spectral window 16 in Table 3 and FIG. 4. Candida pathogens may be distinguished from all bacterial species based on very intense C—O stretching regions in the 1200-1000 cm⁻¹ that show a distinct profile with the band at 1030 cm⁻¹ more intense than the 1080 cm⁻¹. The opposite is the case for all bacterial species which show more intense 1080 cm⁻¹ band form DNA. Other species of bacteria may be distinguished by the different combinations of spectral windows detailed in Table 3. The spectral windows may be classified automatically by feature selection algorithms including random forest and other variable selection methods as described below.

TABLE 3

| PATHOGEN | Spectral window | | | | |
|---|---|---|---|---|---|
| Gram-positive | 22 | 17 | 18 | 19 | 22 |
| Gram-negative | 16 | 17 | 18 | 19 | 22 |
| Klebsiella pneumoniae (−) | 16 | 21 | 16 | 18 | 19 |
| Escherichia coli (−) | 22 | 16 | 17 | 20 | 21 |
| Pseudomonas aeruginosa (−) | 16 | 21 | 16 | 18 | 19 |
| Staphylococcus epidermidis (+) | 22 | 18 | 19 | 22 | 20 |
| Streptococcus dysgalactiae (+) | 22 | 21 | 20 | 19 | 18 |
| Staphylococcus capitus (+) | 22 | 20 | 21 | 19 | 17 |
| Enterococcus faecalis (+) | 22 | 20 | 19 | 18 | 16 |
| Staphylococcus aureus (+) | 22 | 21 | 19 | 18 | 20 |
| Hafnia alvaris (−) | 16 | 17 | 22 | 21 | 19 |
| Stenotrophomonas maltophila (−) | 16 | 17 | 22 | 21 | 19 |
| Enterococcus faecium (+) | 22 | 21 | 19 | 18 | 17 |
| Candida parapsilosis (yeast) | 22 | 21 | 19 | 20 | 18 |

FIG. 5 illustrates Raman spectra of ten common pathogens; Escherichia coli 502 (Gram−), Klebsiella pneumoniae 504 (Gram−), Pseudomonas aeruginosa 506 (Gram−), Enterococcus faecalis 508 (Gram+), Enterococcus faecium 510 (Gram+), Staphylococcus aureus 512 (Gram+), Staphylococcus capitus 514 (Gram+), Staphylococcus epidermidis 516 (Gram+), Strep. dysgalactiae group organism 518 (Gram+) and Candida parapsilosis 520 (Yeast) representing the group of yeast, Gram positive and Gram negative bacteria.

FIG. 5 illustrates the spectral ranges used for analysis: 3050-2800 cm⁻¹ (11); 1750-1500 cm⁻¹ (12); 1500-200 cm⁻¹ (13); 1200-900 cm⁻¹ (14); and 900-700 cm⁻¹ (15). The spectral range between 2800-1750 cm⁻¹ contains no bands of biological origins and is not useful for the diagnostic modeling. The spectral range below 700 cm⁻¹ contains weak bands subject to problems with measurement signal to noise ratio and may not be reliable in terms of diagnostic modeling. The spectral range above 3050 cm⁻¹ contains overtone bands related to bands in the lower wavenumber region that may or may not contribute to the classification performance. Combinations of these regions may be used in modeling or alternatively the spectral regions between 3100-2800 cm⁻¹ and 1800-600 cm⁻¹ may provide optimal modeling with sensitivities and specificities approaching 100%.

Table 4 shows the species of a set of pathogens and a set of Raman spectral windows for distinguishing gram-positive from gram-negative bacteria along with spectral windows for unique pathogen identification using multivariate methods. The spectral windows include: spectral window 11 CH stretching region mainly from lipids and proteins modes (3100-2800 cm⁻¹); spectral window 12 Protein and lipid modes (1750-1500 cm⁻¹) with contributions from cytochrome at 1585 cm⁻¹; spectral window 13 cytochrome and lipid modes (1500-1200 cm⁻¹); spectral region 14 carbohydrate and protein region including phenylalanine mode 1004 cm⁻¹(1200-900 cm⁻¹); spectral region 15 nucleic acid region 900-700 cm⁻¹. The spectral windows decrease in relative importance from left-to-right in Table 4. It should be noted that combinations of two or more windows may improve pathogen identification (e.g., utilising the regions 3100-2800 cm⁻¹ and 1800-600 cm⁻¹). Spectral window numbers are correlated with the shaded areas in FIG. 5.

TABLE 4

| PATHOGEN | Spectral window | | | | |
|---|---|---|---|---|---|
| Gram-positive | 14 | 11 | 12 | 15 | 13 |
| Gram-negative | 14 | 11 | 12 | 15 | 13 |
| Escherichia coli (−) | 15 | 14 | 12 | 11 | 13 |
| Klebsiella pneumoniae (−) | 15 | 13 | 12 | 11 | 14 |
| Pseudomonas aeruginosa (−) | 14 | 13 | 15 | 12 | 11 |
| Enterococcus faecalis (+) | 15 | 14 | 11 | 12 | 13 |
| Enterococcus faecium (+) | 14 | 13 | 15 | 11 | 12 |
| Staphylococcus aureus (+) | 15 | 14 | 12 | 11 | 13 |
| Staphylococcus capitus (+) | 14 | 13 | 12 | 11 | 15 |
| Staphylococcus epidermidis (+) | 15 | 14 | 12 | 11 | 13 |
| Streptococcus dysgalactiae (+) | 12 | 13 | 14 | 11 | 15 |
| Candida parapsilosis (yeast) | 15 | 13 | 11 | 12 | 14 |

Using the spectral windows above for the IR or Raman, the detection of a pathogen may involve a multi-class classifier comprising of two or more spectral windows provided above, wherein the contribution of two or more spectral windows comprise different weights, which may differ ordinally according the spectral windows provided in Tables 3 and 4. For example, for E. coli in Table 3, the weighted contribution from an absorbance within or overlapping with the range of 1200-800 cm⁻¹ is greater than or equal to the weighted contribution from an absorbance within or overlapping with the range of 3100-2800 cm⁻¹, which is greater than or equal to the weighted contribution from an absorbance within or overlapping with the range of 1750-1700 cm⁻¹, which is greater than or equal to the weighted contribution from an absorbance within or overlapping with the range of 1450-1400 cm⁻¹ which is greater than or equal to the weighted contribution from an absorbance within or overlapping with the range of 1300-1200 cm⁻¹. For classifiers that include less than all of the spectral windows, the ordinal weighting of the remaining spectral windows may be maintained. Also, for some classifiers, different features from the same spectral window may have a different weight. For example, with Klebsiella and Pseudomonas in Table 3, the area under the curve of the absorbance at 3100-2800 cm⁻¹ may be weighted differently from the absorbance waveform shape at 3100-2800 cm⁻¹.

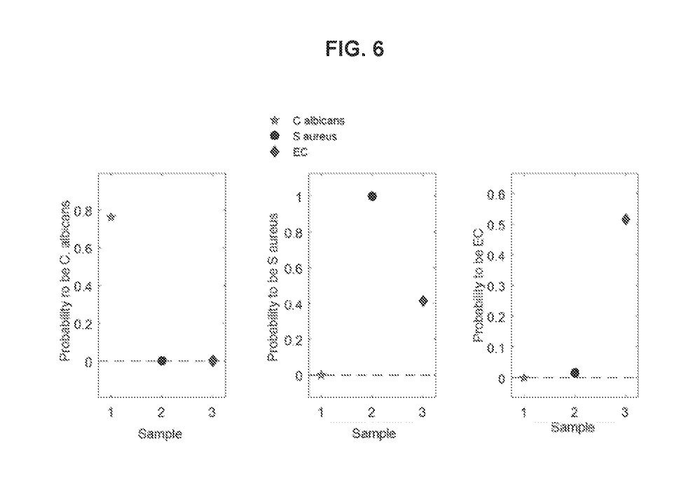

FIG. 6 depicts an example of this multi-class classification using soft independent modeling Soft Independent Modeling of Class Analogy (SIMCA). A model is constructed using a calibration dataset of spectra of dried bacteria in an ATR crystal, containing spectra of Candida albicans (12), Staphylococcus aureus (8) and Escherichia coli (5). In some variations, the calibration dataset of spectra are generated from samples using the same biofluid sample preparation procedure as described herein, e.g., the bacteria is filtered, centrifuged, washed and/or reconstituted with a pre-specified amount of water. The model is tested with one independent sample from each pathogen species. FIG. 6 shows the probability of each test sample to be classified in each species. As can be seen, in all cases the probability of being predicted in the actual class is significative higher than been classified in an incorrect class for the three samples.

### Indirect Detection of Pathogens Using Particle Method

In some variations, silica/silicon particles may be used to trap or aggregate bacteria and the particles may be used as markers to detect the presence of pathogens. The particles may vary in size from about 0.1 micron to about 20 micron. Other nano or micron sized particles made of plastics, metals and non-metals may also be used to trap pathogens (e.g., silver, gold, carbon either functionalised or non-functionalised with molecules that bind to the bacterial or fungal cell wall).

The methods described herein may use either wet or dry serum samples. In some variations, the serum sample may be wet, that is, in liquid form, and may optionally contain a solvent that is naturally occurring, such as water or deliberately added, such as methanol. In some variations, the serum sample may be dry, and may optimally comprise a circle having a diameter of about 0.5 cm. A ‘dry’ serum sample may be formed by collecting a predetermined amount of serum sample (i.e. 10 μL), placing the sample on an appropriate material, and air-drying the sample.

Prior to measuring wet and dry samples, a pre-concentration step may be performed using gel tubes. Gel tubes or serum separating tubes (SST) may comprise a gel separator to improve separation of serum from blood cells. This may concentrate pathogens such as bacteria and fungi, which has the beneficial effect of increasing the sensitivity of infrared spectroscopy for the detection of the pathogens.

Blood from a patient suspected of having sepsis may be separated in serum and cellular fractions may be formed by centrifugation (e.g., at about 3500 r.p.m. for about 10 minutes) using an SST. The pathogen cells may be concentrated in the serum directly above the gel layer in the SST. The silica coating particles may be trapped in the layer directly above the gel. A pipette may draw an aliquot of the serum containing concentrated pathogen cells by placing the pipette tip as close as possible to the gel layer and withdrawing about 20 μL of serum which may be used for infrared or Raman measurement. The gel may have a very specific spectral characteristic and may neither dissolve in the serum nor transfer into it. Therefore, gel contamination is readily apparent in the spectra and another sample may be collected immediately if contamination occurs.

FIG. 8 illustrates a method of developing data models and a cloud based diagnostic platform for the identification of pathogens that cause sepsis in a serum. Serum or plasma may be prepared from venous blood taken from patients 802. Bloodstream pathogens may be isolated from the serum or plasma 804, Infrared or Raman spectra may be acquired using instruments based remotely 806. Classification models may be generated 808 for the following bloodstream pathogens: Candida albicans; Klebsiella pneumonia; Escherichia coli; Pseudomonas aeruginosa; Staphylococcus epidermidis; Streptococcus dysgalactiae; Staphylococcus capitus; Enterococcus faecalis; Staphylococcus aureus; Hafnia alvei; Stenotrophomonas maltophila; Enterococcus faecium; and Candida parapsilosis.

Spectra may be pre-processed 810 on a remote (e.g., cloud-based computer). Spectra may be classified (812) as fungus or bacteria, gram-positive or gram-negative bacteria, and to a particular pathogenic species using a diagnostic algorithm employing the classification models 808. The diagnosis may be transitioned from the remote computing network to the user at the measurement location via local computing network 814.

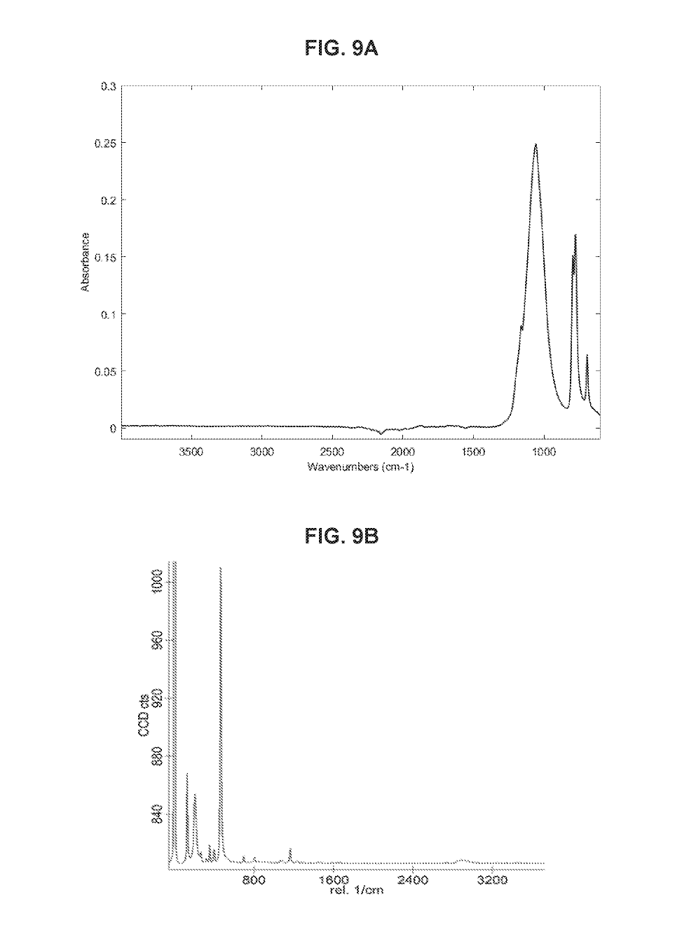

FIGS. 9(A) and (B) illustrate ATR-FTIR and Raman spectra of pure particles from SST tube.

In some variations, the methods discussed herein may be carried out in less than 10 minutes, typically about 5 minutes, including serum separation. By comparison, conventional sepsis detection methods may take about 3 hours or more. The rapid speed of detection according to the present technology is significant given the acute nature of sepsis. In some circumstances, a delay of 3 hours may significantly affect patient outcomes.

In some variations, the generation of spectral models, preparation of calibration spectra from known blood, serum or other biofluid samples known to positive or negative for a specific disease agent may be performed in a laboratory. Once these are generated, the results may be transmitted to an authorized user (e.g., physician, patient, technician).

In some variations, a kit for ATR spectroscopy may comprise a sterile SST tube for deriving serum from whole blood, a sterile syringe filter or ultra-filtration device, a sterile microcentrifuge device, and a pipette with disposable sterile tips. In some variations, a kit for Raman spectroscopy may also include a Raman grade crystalline substrate such as CaF2. In some further variations, the kit may further comprise particles to be added to the biofluid sample for pathogen capture as described above.

### Systems for the Detection, Quantification and Identification of a Pathogens

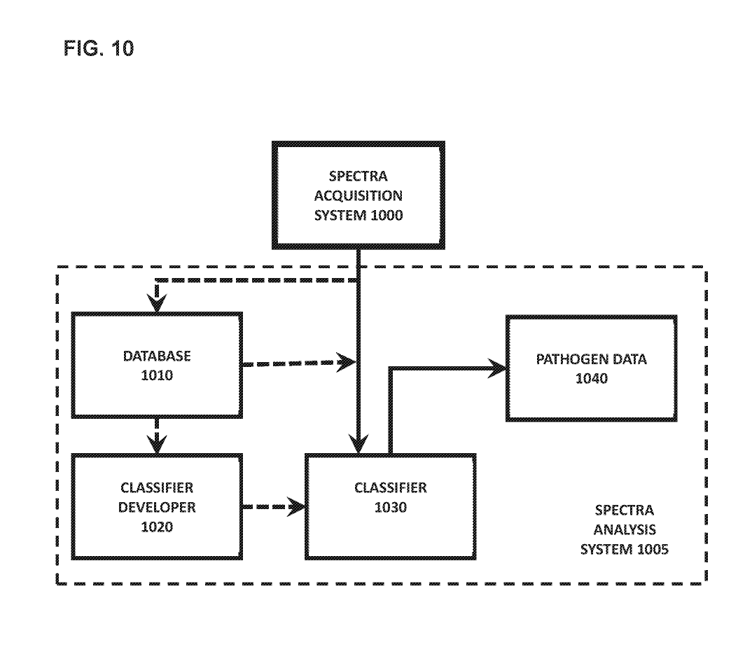

FIG. 10 is a block diagram overview of a system for detecting pathogens, and optionally including a classifier developer to generate new classifiers to detect new pathogens or existing pathogens collected and processed using new processing procedures. The spectra acquisition and validation system 1000 (e.g., spectrometer) may generate a spectrum, which may be transmitted to a spectral analysis system 1005 and stored in a reference database 1010. Additional information, e.g. from blood cultures and/or manual peripheral smear reviews may also be transmitted to the database 1010, either concurrently with the spectra transmission, or at a later time and correlated to the previously transmitted spectra from the same event or patient. Certain data validation processes may be located in the spectra acquisition system 1000, while in other systems the validation processes may be located in the spectra analysis system 1005.

The analysis system 1005 further comprises a classifier developer 1020 may be configured to generate a classifier 1030. The classifier developer 1020 may be configured to search or select new spectra datasets in the reference database 1010 and determine whether there is sufficient data to generate a new classifier, and/or may provide a prediction of the number of additional samples of the suspected new pathogen to achieve the desired level of confidence, sensitivity, and/or specificity desired. The classifier developer 1020 may create a set of selectable classifiers and select classifier, which is then used to generate pathogen data 1040 (e.g., presence/amount/species of a pathogen) from a spectrum of the sample generated from the spectra acquisition system 1000.

Such as system, which is also capable of performing biofluid analysis to identify pathogens, is contrasted with diagnostic systems where the classifiers 1030 have already been determined and the analysis system 1005 may be pre-configured with classifiers 1030 without a classifier developer 1020. In this latter embodiment, spectra transmitted from the acquisition system 1000 to the analysis system 1005 may be stored in the database 1010 or may be sent directly to the classifier 1030 for analysis. This latter embodiment may be used in integrated spectrometer and spectral analysis systems, which may be used at remote locations where the systems may or may not have remote communication modules or assess to a communication network, or where classifier development is not performed.

In some variations, If the spectra are meant to be employed by the classifier developer 2020 in the generation of the classifiers, and the features to predict the sample are known, and the spectra acquired are then transformed from the format of the spectral acquisition system 1000 to a format readable by the classifier developer system 1020 and transmitted to a database 1010 of spectra inside the classifier developer along with the reference data. If the spectra are meant to be classified by the classifier 1030, then the spectra are transformed from the format of the spectral acquisition system to a format readable by the classifier 1030. In some variations, the data format usable by the developer 1020 and the classifier 1030 is the same.

A set of sera or other biofluid samples and their reference data, i.e., whether they are positive or negative for a given pathogen, may be collected and processed using the system 1000. IR spectra of the serum samples may be recorded by the spectra acquisition system 1000 and used to create a matrix X (n=number of samples×v=number of wavenumber values) and a vector of reference data Y (n×1).

The generation of Raman spectra may use a spectral resolution between about 0.5 cm⁻¹ to about 16 cm⁻¹ and in the spectral range of about 20 cm⁻¹ to about 4000 cm⁻¹. Alternatively, for infrared measurements, a set of 1 to about 4000 discrete wavenumbers may be generated in the range 600 to 4000 cm⁻¹. The spectrum may be generated by co-adding the signal over a time interval in the range 0.001 seconds to 10 minutes. A Raman spectrum may be generated using lasers with different excitation energies with wavelengths in a range between 200 nm and 1400 nm.

### Pre-Processing

The noise of the spectra may be reduced by smoothing the spectra with a filter. In some variations, the Savitzky-Golay filter may be used for fitting the spectrum vector on a window of 3 to 30 points to a polynomial of 1^{st} to 6^{th} orders range. A derivative of the spectra may be calculated using the Savitzky-Golay filter, with the 3 to 30 points spectra window and a 1^{st} to 6^{th} polynomial fit. For example, the 1^{st} to 6^{th} derivative of the fitted polynomial may be calculated. Correction of baselines may be performed fitting the spectra to a polynomial of 1^{st} to 6^{th} degree polynomial in the regions where there is no contribution of the sample to the signal, and then subtracting it from the original polynomial in the whole region. The spectral signals may be normalized by adjusting them to correct differences in path length. Normalization may be performed by dividing the spectral values between one of a range, an integrated area, the average of the spectra, and the standard deviation of the spectra (e.g., Standard Normal Variate). In some variations, the spectral signals may be adjusted to obtain consistent differences between the maximum and minimum of the spectra (e.g., min-max normalization).

Non-informative parts of the spectra may be removed in order to improve the accuracy, efficiency and/or speed of the classification. In particular, it is preferable to use a limited number of wavenumber values (selected spectral windows as shown in Table 3).

### Quality Control

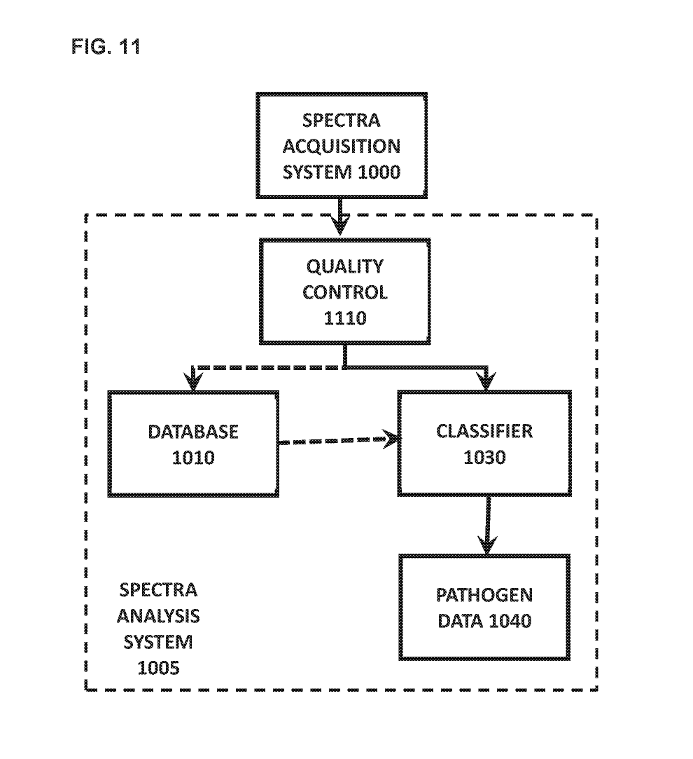

FIG. 11 describes the general process the spectral acquisition and validation system 1000 that may generating the spectrum 1100, performing additional quality control and data manipulation of the spectrum data for storage in a database 1120 and analysis by the classifier 1130. (e.g., format the data for the identification and quantification system). Although FIG. 11 depicts the quality control analyser 1110 as part of the spectra analyser 1005, all or a portion of the quality control analyser may also be performed in the acquisition system 1000.

The spectrometer data may be classified as insufficient due to, for example, poor contact between the sample and the ATR crystal in the spectrometer. The quality control process may detect excesses (or deficiencies) of the different components and interferences in the sample. The component's relative concentration may be calculated and this concentration may be compared to a threshold value. For example, a distribution of relative concentration values of the component may be generated by the controller. Then the portions of the distribution that tail off at the upper and lower ends can be used to define the threshold. If the relative concentration of the component is outside the threshold, the data may be classified as failing quality control.

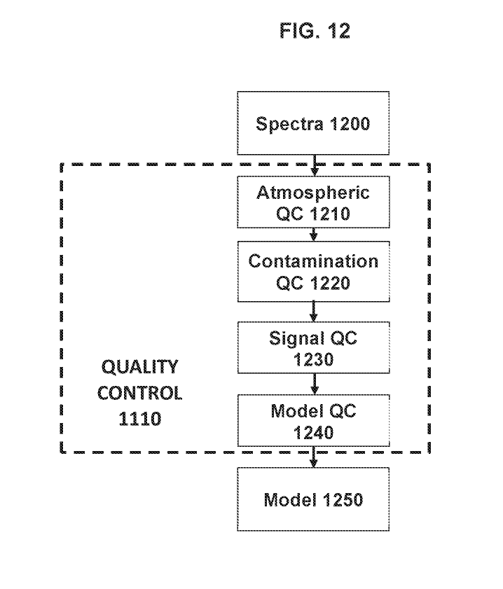

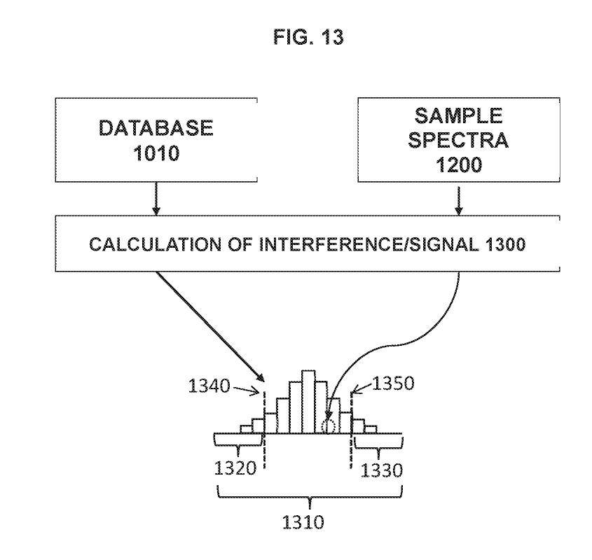

Validation of the spectra may be performed prior to inclusion into one of the aforementioned models. This ensures that an acquired spectrum has features similar to the features included in the model. It also ensures that technical issues are not going to interfere in the extraction of information from the model. For example, FIGS. 12 and 13 represent exemplary methods of quality control that may be incorporated into the quality control process 1110 as depicted in FIG. 11 to determine whether the spectra have the correct signal and free from contributions from contaminants, for example.

In FIG. 12, spectra 1200 that are received are checked for the atmospheric contaminants 1210, sample contaminants 1220, and signal quality 1230. For example, for the atmospheric contaminants 1210, fluctuations of IR or Raman active atmospheric vapours between the background and sample measurements may create negative and positive bands, which may be detected using positive and negative thresholds. Sample processing related contaminations 1220 may include detection of a solvent (water or other organic or inorganic solution) that has not been properly eliminated or silica or other particles have been contaminated the sample. Poor signal quality 1230 may be due to poor contact of the sample with the ATR crystal or a non-optimal focus when using a Raman microscope or other Raman measurement device. This may be checked by looking for wideband attenuation of the absorbance signals.

Another quality control check 1240 may be associated with the model 1250 and rely on the measurement of the distance between the sample and the calibration samples in terms of the modeling. For example, Hotelling's T² and SQ residuals on a PLS-DA may be used with a 95% confidence interval. In some variations, quality control for a spectrum may be carried out in the sequence of atmospheric interference (water), solvent (water or methanol), sample, and distance to the model.

Once the received spectra 1200 has undergone one or more of the quality check processes, the spectra 1200 may then be provided to the model 1250 for further processing. In some variations, the QC may provide a binary outcome whereby the spectra 1200 and/or model 1250 passes all or a sufficient number of the QC checks so that the spectra 1200 can then be processed by the model 1250, or failed and an error message is generated and sent to the analysis system 1005 and/or the acquisition system 1000. In other QC systems, a multi-level or branched QC system is provided, such that where the spectra 1200 is processable by the model 1250 but certain confidence, sensitivity and/or specificity standards may not be met or are at risk of bias, the analysis system 1005 and/or the acquisition system 1000 may be provided with one or more warning or modifier messages along with the diagnostic output from the analysis system 1005.

FIG. 13 depicts another exemplary quality control process that may be provided in the analysis system, which utilises only the database 1010, that is, independent of the model. The process monitors excesses (or defects) of different components and interferences pertaining to the sample spectra 1200. The component relative concentration is calculated and compared with a threshold value based on an absorbance criterion for infrared spectra and an intensity criterion for Raman spectra. For example, a distribution 1310 of relative concentration values of the component may be stored in the database 1010. Then the portions of the distribution that tail off at the upper and lower ends, 1320, 1330 may define the thresholds 1340, 1350. If the relative concentration of the component is outside the threshold, the analysed spectrum fails the quality control process. For infrared spectroscopy, the threshold may be less than 2×10⁻³ absorbance units and for Raman, the threshold may be 50 counts based on the strongest band in the spectrum.

### Classifier Developer

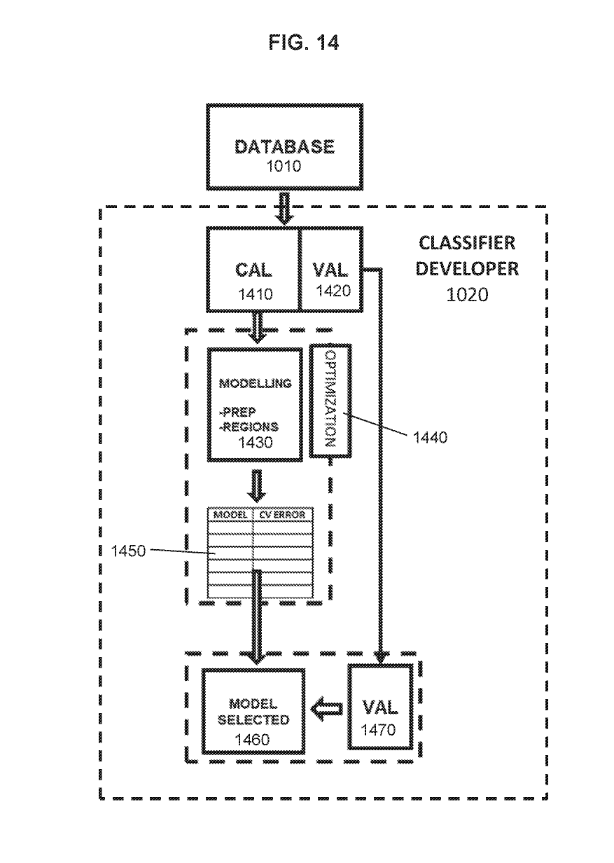

Referring back to FIG. 10, the optional classifier developer 1020 may receive data from the spectral database 1010 and spectra system 1000 generate a classifier 1030 for an unknown sample. Unknown samples are samples whose reference values are unknown and therefore may be transmitted to the classifier developer 1020 generate a new classifier 1030 if the database 1010 contains sufficient data. The block diagram in FIG. 14 depicts the process to generate a model which identifies and quantifies a pathogen from the FTIR or Raman spectrum. The data received from the database 1010 may be received by the developer 1020 and split into calibration 1410 and validation 1420 datasets. The calibration dataset 1410 is used for generating and optimising the model. The optimization aims to compare the performance of several classifiers by varying the following: i) the statistical method used (e.g., PLS-DA, SVM and other available methods explained in greater detail below); ii) the internal parameters of the statistical method, such as the number latent variables in PLS-DA, or the cost parameter in the SVM calibration; iii) the spectral preprocessing used and iv) the spectral region used. The optimisation process 1440 entails the creation of several classifiers using different statistical methods, as described in greater detail below, including different internal parameters of these statistical methods, preprocessing and spectral regions described and variable selection described below. For each different statistical method, the set of parameters 1430 selected for the models may be selected according to the expertise of the user or employing random combinations of the parameters, or may be performed automatically without user input. For each model, the performance of the model is assessed using error parameters such us cross validation or bootstrapping procedures. The classifiers are then sorted in the table 1450 according to their performance and the classifier which performs the most robust classification (the classifier achieving the lower error) is selected. Cross validation or other error measurement may be applied to select the model or classifier 1460. Then the performance of the selected model may be tested with the validation dataset 1470.

A relevant pattern may be found through a model expressed in terms of the range of operations expected to be performed by the classification function (that is the possible g(x) of f=g(x)). An iterative mathematical process may be used to establish g(x). In some variations, the model may comprise a function Y=f(X_{spectrum}) corresponding to the spectrum X of a biofluid and some attribute of the biofluid sample Y. This Y attribute may be any information of the sample. For example, Y may correspond to the presence of a pathogen as pathogen signal bands, which are different from the noise baseline or other biofluid components (e.g., Y=1 or 0 for detectable and not detectable, respectively). Y may correspond to the classification of the pathogens in a library of pathogens based on spectral similarities (e.g., Y=1, 2, 3 . . . n, representing each number of a class). Y may correspond to the amount of bacteria dried on the crystal. In this case, Y may be a numerical value representing the amount of bacteria.

Spectra containing and excluding one or more pathogens may be inputted to the classification model, and used to learn the characteristic spectral components of the spectra for each class (e.g., positive for presence of the disease, negative or unknown) to form a calibration matrix. From this a set of mathematical operations can be applied to the spectrum (a vector of absorbance values or Raman intensity values) for a new biosample to generate a value that determines if the spectrum can be classified as positive or negative.

A model may be specific for one or more pathogens based on the spectral windows shown in Tables 3 and 4 or alternatively the entire spectral range 4000 cm⁻¹ to 100 cm⁻¹.

Referring still to FIG. 14, in some variations, the classifier development process may comprise receiving calibration infrared spectra 1410 representative of serum samples comprising the pathogen and serum samples absent the pathogen agent. The calibration spectra may comprise one or more spectral components, each component having a wavenumber and absorbance value. A function (e.g., an mathematical operation) may be generated Y=f(X_{spectrum}) establishing the relationship between the spectrum X of the serum or other biofluid samples and the spectral components of the biofluid samples Y that identify the disease agent.

Once an appropriate y=fselected(x) is established with known error limitations, it can be applied to a spectrum of a new serum or other biofluid sample from a patient to determine a response (positive and negative).

It will be readily apparent to the person skilled in the art that the processes described herein may be iterative, but selection of the parameters may also be carried out by various other approaches, combinations, and permutations of approaches including using the knowledge the character of the analytical problem. For example, if the model is non-linear, PLS-DA is not used or alternatively the CO₂ region is not used for classification. Iterative procedures may also be used. For example, a genetic mathematical operation in the variable selection such as Random Forest feature selection may be used.

The quality of classification of the function selected in the modeling step may be evaluated using an independent test of samples. That is, the validation or test spectra 1470 of the validation samples 1420 may be introduced as an input in the function of the classifier selected 1460 and the output \tilde{Y}ₜₑₛₜ is compared with the reference data Yₜₑₛₜ. The error value is the expected error of the classification. If through this process the model is assumed to be of sufficient quality, the model may be ready to be used for classification with a new patient serum, blood or other biofluid sample of unknown disease status.

Reference samples may be used to calibrate the classifier as a set of reference data. The references samples may undergo pre-processing and quality control checks as described elsewhere herein. The reference data (e.g. presence/amount/species of a particular pathogen) may be stored in the database.

Extraneous external sources of variation may be removed from the spectra prior to modeling to enhance the differences of the spectra connected with the bands of interest. The average spectra of the calibration data (mean centering) may be subtracted for enhancing the differences between spectra. The variables may be scaled by dividing each variable between the standard deviation of the variable across the calibration set (Autoscale function).

In some embodiments, Partial Least Squares Discriminant Analysis (PLSDA) may be used to model the calibration data by creating a series of latent variables (LVs) (Between 0 and the total number of samples or variables) which captures the variability of the spectra of the calibration sample dataset and correlates it with a vector Y which contains ones for the positive and zeros for the negative samples. Cross Validation (CV) is generally used for establishing the optimal number of LVs. In some variations, Artificial Neural Networks (ANN) may connect an input layer of elements (i.e. spectral variables) with a hidden layer of elements which perform operations with the variables. The results of these operations are weighted as described previously and may be subjected to further transformations in other layers. At the end, the layers may be connected in an output layer, which provides a result corresponding to the presence of a pathogen. In some variations, a Support Vector Machines classifier (SVMc) may use a kernel for mapping the original input variables into a high dimensional space where the classes are linearly separable by a hyperplane using supported vectors. The user may select a kernel (e.g., Linear, polynomial, Gaussian radial basis function (RBF)) and then optimize the model for different parameters, including the cost parameter C or the gamma parameter if Gaussian RBF is selected.

Spectra with, and without the pathogen may be inputted to the model, to learn the characteristic spectral components of the spectra for each class (e.g., positive for presence of the disease, negative or unknown) to thus form a calibration matrix. From this is provided a set of mathematical operations that can be applied to the spectrum (a vector of absorbance values or Raman intensity values) for a new biosample to generate a value that determines if the spectrum can be classified as positive or negative.

### Pathogen Quantification

In some variations, the amount of bacteria dried onto the crystal may be a feature. In this case, Y is a numerical value representing the amount of bacteria. Quantification of the pathogen may provide a prediction of the amount of colony forming units (CFUs) in the sample. A number of functions may be used to quantify the pathogen, including PLS regression (PLSR) that is a model equivalent to PLSDA, but which performs a regression. Instead of ones and zeros, the Y vector contains the concentration of pathogens. The optimization of the LVs is similar to the PLSDA optimization according to the CV. An ANN may also be used for the prediction of a continuous variable. In some variations, Support Vector Machines regression (SVMr) may be used to calculate the concentration, and may be optimized in the same way as the SVMc, except that a parameter epsilon should be optimized.

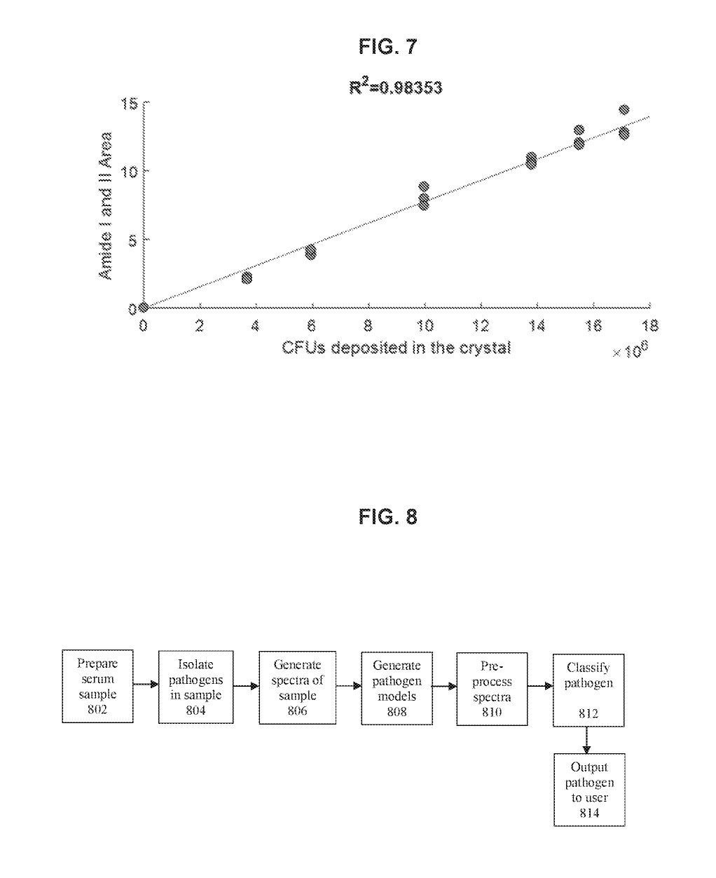

FIG. 7 represents a univariate model for the quantification of the bacteria based on the integration of the amide band, e.g. certain peaks or AUCs of the absorbances from 1700-1500 cm⁻¹. The Limit of Detection (LOD) and limit of quantification, established as the concentration corresponding to 3 and 10 times the standard deviation of the signal of the blanks (n=9) were 7730 and 22600 cfu in the crystal, respectively.

### Processing System

A spectroscopy analysis and processing system may comprise a controller in communication with one or more spectrometers. The controller may comprise one or more processors and one or more machine-readable memories in communication with the one or more processors. The processor may incorporate data received from memory and operator input to control the spectroscopy processing system. The inputs to the controller may be received from one or more machine generated (e.g., spectrometers) and/or human generated sources (e.g., user input). The memory may further store instructions to cause the processor to execute modules, processes and/or functions associated with the processing device, such as the methods described herein. The controller may be connected to the one or more spectrometers by wired or wireless communication channels. The controller may be configured to control one or more components of the spectroscopy processing system including the network interface and user interface.

A controller may be configured to perform processing and/or analysis of spectroscopy data, such as determining spectroscopy data quality, as described elsewhere herein. The system may provide centralized data collection and standardized spectroscopy signal processing across a plurality of remote locations. The system may also allow an authorized user to access and review patient study results and perform additional analysis. For instance, different levels of patient results may be available to one or more patients, caretakers, healthcare providers, health plans and authorized internal and/or external users via a web-based interface. Record keeping, security and consistency may thus be improved when data processing and data storage may be centralized at a spectroscopy processing system. This also allows trained personnel such as infectious disease specialist or laboratory technicians that manually process and review spectroscopy data to be provided access at a central location, further increasing efficiency and cost savings.

A controller may be configured to import and selectively store data from a spectrometer. For example, the controller may be configured to determine the spectroscopy data quality by executing a quality control process prior to performing any additional analysis. The quality control process may include a variety of features. For example, atmospheric contaminant identification involves identification of atmospheric contaminants that can affect the assay. Fluctuation of IR or Raman active atmospheric vapors between the background and sample measurements may cause negative and positive bands which may be detected by using positive and negative thresholds. Contaminant identification may seek to identify one or more solvents and/or particles (e.g., silica) used and/or present in the preparation of the sample (water, MeOH) that have not been properly eliminated.

Referring back to FIG. 10, the spectroscopy analysis system 1005 may comprise a set of reference models stored in a relational database 1010 that may be used to generate classifiers and/or store classifiers to assess biofluid samples. The format of the database and the data structures it contains may correspond to the type of spectrometer and its output format. In some variations, the reference models may be developed by obtaining a sample set. A set of sera or other biofluid samples and corresponding reference data (that is, whether they are positive or negative for a given pathogen) may be recorded and/or provided. IR spectra of the serum samples may be recorded and used to create a matrix X (n=number of samples, v=number of wavenumbers) and a vector of reference data Y (n×1).

The IR spectra may be generated with a spectral resolution in the range of about 0.5 to about 1000 cm−1 in the spectral range of about 20 to about 4000 cm−1. Alternatively, a set of 1 to about 4000 discrete wavenumbers may be generated in the range of about 20 to about 4000 cm−1. The spectrum may be generated by co-adding between 1 and 1064 scans. A background of air or solvent may be generated under the same experimental conditions before acquiring the spectrum of the sample.

The Raman spectra may be generated with a spectral resolution in the range of about 0.5 to about 1000 cm−1 in the spectral range of about 20 to about 4000 cm−1. Alternatively, a set of 1 to about 4000 discrete wavenumbers may be generated in the range of about 20 to about 4000 cm−1. The spectrum may be generated by co-adding the signal over a time interval in the range of about 0.001 seconds to about 10 minutes. The Raman signal may be generated using lasers with different excitation energies with wavelengths in a range of between about 200 and about 1400 nm.

The controller may execute a variable selection process to identify the features of the sample and isolate them to aid classification. The variable selection process may include the steps of removing non-informative parts of the spectra in order to improve the accuracy, efficiency and/or speed of the classification. In particular, a limited number of wavenumbers (selected spectral windows) may be included that are relevant to a particular disease. Multiple spectral windows may be processed sequentially or simultaneously. Processing of two or more selected spectral windows may improve the accuracy of results obtained using the model. An example of the spectral windows to be selected is available in Table 3 and Table 4. For the best sensitivity and specificity the entire spectral range between 3100-2800 cm⁻¹ and 1800-600 cm⁻¹ can be modeled and predicted.

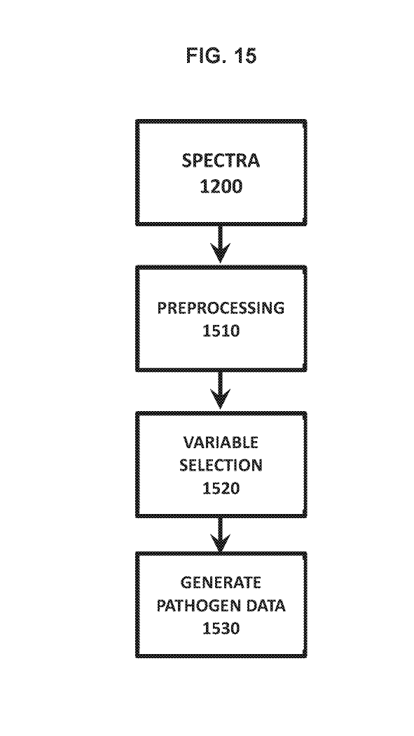

The spectroscopy processing system may comprise a classifier which imports and/or stores the pre-processed data for comparison against a set of models (e.g., from a database) to determine the presence of a pathogen. A pathogen may be further identified using a classifier. As shown in FIG. 15, the process may include acquiring the spectrum 1200, performing preprocessing 1510 on the spectrum data 1200, and performing variable feature selection on the spectrum data 1520. Pathogen data 1530, e.g. presence and identification of the pathogen, may be predicted using the spectrum data 1200 with a selected model, and the pathogen load may be quantified.

In some variations, a y=fselected(x) may be established with known error limitations and then applied to a spectrum of a serum or other biofluid sample from a patient to determine a response (positive and negative).

The model generated may be linear or non-linear and may be compared to a serum or other biofluid sample of unknown disease status. For example, a linear model may be created based on discriminant analysis by a partial least squares algorithm (PLS-DA) to provide a regression vector of weights (W) of each wavenumber (i): W=(w1, w2, w3 . . . wi)

Variable or feature selection 1520 involves removal of the non-informative parts of the spectra 1200 to improve the accuracy, speed and/or efficiency of the classification, as only a limited number of wavenumbers (selected spectral windows) may be relevant to a particular or suspected disease. There are various ways to select those wavenumber values, from the straightforward selection of the regions of interest to complex iterative selections such as genetic mathematical operation such as Random Forest Feature selection.

For example, one or more of the wavenumber ranges or subranges between the spectral windows described above may be omitted from analysis. Omitted wavenumber ranges or cutoffs may include wavenumbers below 700 cm−1, 1300 to 1400 cm−1, 1450 to 1500 cm−1, and/or 1750 to 2800 cm−1, or subranges therefore, for ATR spectroscopy.

In another example, the spectra may be characterised as a vector of absorbance values X=(x1, x2, x3 . . . xi). The final outcome may be calculated by multiplying the regression vector W(i) by the absorbance values of the spectrum at each absorbance X(i): Y=(w1×1+w2×2+w3×3 . . . wi×i). Y values close to +1 may be assigned to one class (e.g. positive for the disease agent) and Y values close to 0 may be assigned to another class (e.g. negative for the disease agent). It will be appreciated that the cut-off values relating to assignment to one class or the other are arbitrary and is one of the variables that can be optimised, or altered. This might be appropriate for example, if it is preferred to have more false positives than false negatives.

Classification of the pathogen may classify the pathogen depending on one or more characteristics of the pathogen (e.g., Gram positive or negative). Classification may be performed using Soft Independent Modeling of Class Analogy (SIMCA) which performs a Principal Component Analysis (PCA) on each class in the dataset. An optimal number of Principal Components (PCs) may be selected to capture enough variance in each class. By comparing the residual variance of an unknown spectrum to the average residual variance of the spectra of the samples of each class, it is possible to obtain a direct measure of the probability of each sample to be in each class.

In some variations, the performance of all the processing for each dataset is independently investigated. The selection of the best modeling system may be based on the prediction results obtained by cross validation. In some variations, aggregation models may be used. That is, for each sample, each modeling system gives a vote for a class and the final prediction may be obtained by computing the mode of those votes.

It is noted there are no best models established for the diagnosis of a particular pathogen. Depending on different factors inherent in the disease agent, the number of samples and the variability of those samples, some models show better classification performance than others. Accordingly, for each application, a prior deep study of the different modeling possibilities should be performed. Optimally, all the variables involved in the modeling should be established. Other variables that may be optimised to create a useful model include the number of trees in the Random Forest and the number of latent variables in the PLSDA.

In some variations, the presence of a pathogen may be determined by specific pathogen bands which are different from the noise baseline (e.g., Y=1 or 0 for detectable and not detectable, respectively).

In some variations, the classification of the pathogens may use a library of pathogens based on spectral similarities in a library of pathogens where Y=1, 2, 3 . . . n, representing each number a class. A model may be specific for one specific pathogen or combinations of pathogens.

It will be readily appreciated that the method applied may be monitored to ensure ongoing accuracy. This can be carried out using control samples which can be predictive of future changes in the model. The model created and applied to a serum or other biofluid sample of unknown disease status may be linear or non-linear. In some variations, a linear model may be created based on discriminant analysis by a partial least squares algorithm (PLS-DA), to provide a vector of weights of each wavenumber (i), such as a regression vector: W=(w1, w2, w3 . . . wi), which may correspond to the spectral windows in Tables 3 and 4.

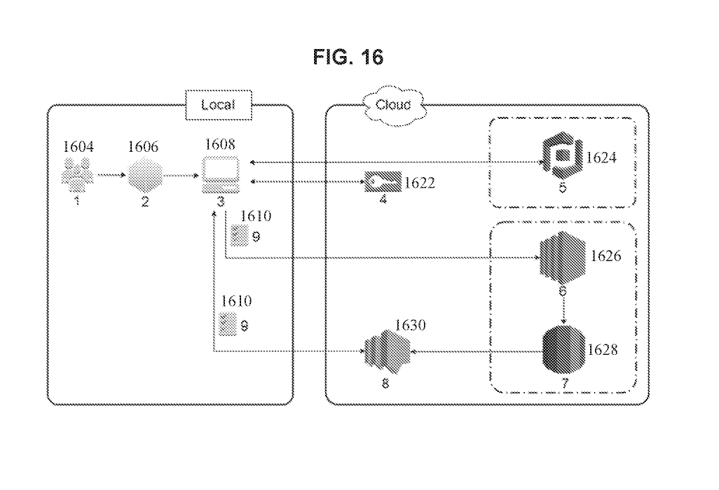

FIG. 16 schematically depicts an exemplary system architecture of a cloud-based spectroscopy system 1600. The system 1600 may comprise local computing network 1602 and remote computing network (e.g., cloud-based system) 1620. The network 1602 is local in the sense that one or more users (e.g., patients, technicians, health care providers) may provide a sample to a spectrometer 1606. The spectrometer 1606 may be coupled to a control system 1608 (e.g., computer systems, computing device). The control system 1608 may be located in the same room as the spectrometer 1606, in an adjacent or nearby room, or tele-operated from a remote location in a different building, city, or country, etc. In some variations, a plurality of spectrometers 1606 and/or control systems 1608 may be provided. The control system 1608 may comprise RF circuitry to communicate with the remote network 1620. In some variations, a secure token service 1622 (e.g., security token) and/or a user authentication service 1624 may be used to secure communication between the local network 1602 and remote network 1620 and prevent unauthorized access to patient data. The token 1622 may include cryptographic keys, passwords, digital signatures, and the like. User authentication service 1624 may include, for example, desktop single sign-on (SSO) and username/password verification.

In some variations, spectral data may be transmitted to a remote server 1626, 1628 for processing of spectral data. For example, server 1626 may perform quality control processing, classification processing, and other processing as described herein on patient spectral data. Database server 1628 may store spectral data, reference models, and other user data. In some variations, the database 1628 may receive processed data from server 1626 and transmit reference data to the server 1626. The servers 1626, 1628 may be provided on the same or different networks.

Communication between the servers 1626, 1628 and the control system 1608 may be secured using a unique identifier 1610 (e.g., username/password, biometric authentication, etc.). In some variations, a secure notification service 1630 may be used to ensure communication between the local network 1602 and remote network 1620 is secured.

The controller may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers, or server computing devices such as routing/connectivity components, portable (e.g., hand-held) or laptop devices, multiprocessor systems, microprocessor-based systems, and distributed computing networks. Examples of portable computing devices include smartphones, personal digital assistants (PDAs), cell phones, tablet PCs, phablets (personal computing devices that are larger than a smartphone, but smaller than a tablet), wearable computers taking the form of smartwatches, portable music devices, and the like, and portable or wearable augmented reality devices that interface with an operator's environment through sensors and may use head-mounted displays for visualization, eye gaze tracking, and user input.

The processor may be any suitable processing device configured to run and/or execute a set of instructions or code and may include one or more data processors, image processors, graphics processing units, physics processing units, digital signal processors, and/or central processing units. The processor may be, for example, a general-purpose processor, Field Programmable Gate Array (FPGA), an Application Specific Integrated Circuit (ASIC), and the like. The processor may be configured to run and/or execute application processes and/or other modules, processes and/or functions associated with the system and/or a network associated therewith. The underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

In some variations, one or more processors may execute the methods described herein in a cloud computing environment or as a Software as a Service (SaaS). For example, at least some of the steps of the methods described herein may be performed by a group of computers in communication via a network (e.g., the Internet) and via one or more appropriate interfaces (e.g., APIs). The cloud computing system may include clients and servers. A client and server are generally remote from each other and typically interact through a communication network. The relationship of client and server arises by virtue of computer programs running on the respective computers and having a client-server relationship to each other.

In some variations, the memory may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. As used herein, database refers to a data storage resource. The memory may store instructions to cause the processor to execute modules, processes and/or functions associated with the spectroscopy processing system, such as spectroscopy data processing, communication, display, and/or user settings. In some variations, storage may be network-based and accessible for one or more authorized users. Network-based storage may be referred to as remote data storage or cloud data storage. Spectroscopy data stored in cloud data storage (e.g., database) may be accessible to respective users via a network, such as the Internet. In some variations, database may be a cloud-based FPGA.

The memory may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. As used herein, database refers to a data storage resource. The memory may store instructions to cause the processor to execute modules, processes and/or functions associated with the processing device, such as spectroscopy data processing, communication, display, and/or user settings. In some variations, storage may be network-based and accessible for one or more authorized users. Network-based storage may be referred to as remote data storage or cloud data storage. Spectroscopy data stored in cloud data storage may be accessible to respective users via a network, such as the Internet.

Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for a specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs); holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, JAVA®, Python, Ruby, VISUAL BASIC®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

A user interface may permit an operator to interact with and/or control the processing system directly and/or remotely. For example, the user interface may include an input device for an operator to input commands and an output device for an operator and/or other observers to receive output (e.g., view patient data on a display device) related to operation of the processing system. In some variations, the user interface may comprise an input device and output device (e.g., touch screen and display) and be configured to receive input data and output data from one or more of the spectrometers, input device, and output device. For example, spectroscopy data generated by spectrometers may be processed by controller and displayed by the output device (e.g., monitor display). As another example, operator control of an input device (e.g., joystick, keyboard, touch screen) may be received by user interface and then processed by controller for user interface to output a control signal to one or more of the processing system and spectrometers.

An output device of a user interface may output spectroscopy data corresponding to a patient, and may comprise one or more display devices. The display device may be configured to display a graphical user interface (GUI). A display device may permit an operator to view spectroscopy data and/or other data processed by the controller. In some variations, an output device may comprise a display device including one or more of a light emitting diode (LED), liquid crystal display (LCD), electroluminescent display (ELD), plasma display panel (PDP), thin film transistor (TFT), organic light emitting diodes (OLED), electronic paper/e-ink display, laser display, and holographic display.

Some variations of an input device may comprise at least one switch configured to generate a control signal. For example, an input device may comprise a touch surface for an operator to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies.

A processing system described herein may communicate with one or more networks and spectrometers through a network interface. In some variations, the processing system may be in communication with other devices via one or more wired and/or wireless networks. For example, the network interface may permit the processing system to communicate with one or more of a network (e.g., Internet), remote server, and database. The network interface may facilitate communication with other devices over one or more external ports (e.g., Universal Serial Bus (USB), multi-pin connector) configured to couple directly to other devices or indirectly over a network (e.g., the Internet, wireless LAN).

In some variations, the network interface may comprise radiofrequency (RF) circuitry (e.g., RF transceiver) including one or more of a receiver, transmitter, and/or optical (e.g., infrared) receiver and transmitter configured to communicate with one or more devices and/or networks, RF circuitry may receive and transmit RF signals (e.g., electromagnetic signals). The RF circuitry converts electrical signals to/from electromagnetic signals and communicates with communications networks and other communications devices via the electromagnetic signals. The RF circuitry may include one or more of an antenna system, an RF transceiver, one or more amplifiers, a tuner, one or more oscillators, a digital signal processor, a CODEC chipset, a subscriber identity module (SIM) card, memory, and the like. A wireless network may refer to any type of digital network that is not connected by cables of any kind. Examples of wireless communication in a wireless network include, but are not limited to cellular, radio, satellite, and microwave communication. The wireless communication may use any of a plurality of communications standards, protocols and technologies, including but not limited to Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email (e.g., Internet Message Access Protocol (IMAP) and/or Post Office Protocol (POP)), instant messaging (e.g., eXtensible Messaging and Presence Protocol (XMPP), Session Initiation Protocol for Instant Messaging and Presence Leveraging Extensions (SIMPLE), and/or Instant Messaging and Presence Service (IMPS)), and/or Short Message Service (SMS), or any other suitable communication protocol. Some wireless network deployments combine networks from multiple cellular networks or use a mix of cellular, Wi-Fi, and satellite communication. In some variations, a wireless network may connect to a wired network in order to interface with the Internet, other carrier voice and data networks, business networks, and personal networks. A wired network is typically carried over copper twisted pair, coaxial cable, and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). As used herein, network refers to any combination of wireless, wired, public, and private data networks that are typically interconnected through the Internet, to provide a unified networking and information access system.

Processing of the spectroscopy data or recording may be performed using the hardware described herein using a wired or wireless communication link with the spectrometers at the clinic or laboratory site where the patient is located. The communication between the processing system and the spectrometers may or may not be performed in real-time as the spectroscopy data is received or recorded. The processing may be located in the same housing as the spectrometers, or in a separate housing in the same room or building as the spectrometers. The processing system may also be located in a remote location from the spectrometers (e.g., a different building, city, country).

While this technology has been described in connection with specific variations thereof, it will be understood that it is capable of further modification(s). This application is intended to cover any variations uses or adaptations of the technology following in general, the principles of the technology and including such departures from the present disclosure as come within known or customary practice within the art to which the technology pertains and as may be applied to the essential features hereinbefore set forth.

As the present technology may be embodied in several forms without departing from the spirit of the essential characteristics of the technology, it should be understood that the above described variations are not to limit the present technology unless otherwise specified, but rather should be construed broadly within the spirit and scope of the technology as defined in the appended claims. The described variations are to be considered in all respects as illustrative only and not restrictive.

Various modifications and equivalent arrangements are intended to be included within the spirit and scope of the technology and appended claims. Therefore, the specific variations are to be understood to be illustrative of the many ways in which the principles of the present technology may be practiced. In the following claims, means-plus-function clauses are intended to cover structures as performing the defined function and not only structural equivalents, but also equivalent structures.

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

It is to be appreciated that any discussion of documents, devices, acts or knowledge in this specification is included to explain the context of the present technology. Further, the discussion throughout this specification comes about due to the realisation of the inventor and/or the identification of certain related art problems by the inventor. Moreover, any discussion of material such as documents, devices, acts or knowledge in this specification is included to explain the context of the technology in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

1. A system for detecting a disease agent in a sample derived from a patient biofluid, comprising:
a receiver coupled to a communication network;
a controller coupled to the receiver, the controller comprising a processor and a memory, and the controller configured to:
generate an infrared spectrum of the sample, the sample spectrum comprising one or more sample spectral components, the sample spectral components comprising a sample wavenumber and a sample absorbance value;
provide a set of reference spectral models comprising one or more reference spectral components, the reference spectral components comprising a reference wavenumber and a reference absorbance value, wherein the reference spectral components comprise one or more pathogen characteristics associated with sepsis;
classify the one or more sample spectral components as pathogenic or non-pathogenic using the reference spectral models; and
generate pathogen data using the classified sample spectral components.

2. A system for detection of a disease agent in a sample derived from a patient biofluid, comprising:
a receiver coupled to a communication network;
a controller coupled to the receiver, the controller comprising a processor and a memory, and the controller configured to:
record an infrared spectrum of the sample to the memory, the sample spectrum comprising one or more spectral components, the sample spectral components comprising a sample wavenumber and a sample absorbance value;
provide a set of reference spectral models comprising one or more reference spectral components, the reference spectral components comprising a reference wavenumber and a reference absorbance value, wherein the reference spectral components comprise one or more pathogen characteristics including pathogenic cell quantity;
classify the one or more sample spectral components as pathogenic using the reference spectral models;
calculate a number of pathogenic cells in the sample using the reference spectral models; and
generate pathogen data using the classified sample spectral components and the calculated number of pathogenic cells.

3. A method of screening for a pathogen in a patient, comprising:
extracting a test sample from patient biofluid using a filter;
applying the test sample to an ATR crystal or infrared/Raman substrate;
delivering an electromagnetic beam from the infrared-visible spectrum through the test sample; and
detecting at least one of an absorbance and beam scattering of the test sample to assess the presence of a pathogen.

4. The method of claim 3, wherein extracting the pathogen comprises;
isolating a particulate sample from the biofluid using the filter,
suspending the particulate sample in a solvent,
concentrating the particulate sample in an amount of solvent to form a concentrated test sample.

5. The method of claim 3 or 4, further comprising analysing absorption or scattering of the electromagnetic beam by the sample to identify a pathogen.

6. The method of any one of claims 3 to 5, wherein the electromagnetic beam is an evanescent infrared beam.

7. The method of any one of claims 3 or 4, further comprising identifying a molecular phenotype corresponding to a pathogen type by comparing absorption or scattering of the electromagnetic beam by the test sample with a database or with a spectral model for a pathogen.

8. A method of screening for a pathogen in a patient, comprising:
centrifuging a biofluid sample from the patient in the presence of particles;
delivering an electromagnetic beam from the infrared-visible spectrum through the patient biofluid sample; and
detecting the presence of particles associated with at least one pathogen.

9. The method according to claim 8, wherein a serum separator tube is used to centrifuge the biofluid sample.

10. The method according to claim 8, further comprising:
analysing absorption or scattering of the electromagnetic beam by the sample to detect the presence of particles associated a pathogen.

11. The method according to any one of claims 8 to 10, further comprising:
identifying the pathogen using the absorption or scattering.

12. The method according to claim 11 wherein identifying the pathogen comprises identifying a molecular phenotype which is specific for the pathogen type by comparison of absorption or scattering of the electromagnetic beam by the sample with a database or with a spectral model for the pathogen.

13. The method according to any one of claims 3 to 12, further comprising determining a quantitative concentration of the pathogen in the test sample.

14. The method according to any one of claims 3 to 13 wherein the test sample includes two or more pathogens.

15. The method according to any one of claims 3 to 13 wherein the pathogen is associated with sepsis.

16. The method according to any one of claims 3 to 12, further comprising quantifying a pathogenic load in the test sample by comparing absorption of the electromagnetic beam by the test sample with calibration models to quantify the number of pathogen cells present in the test sample.

17. The method according to claim 16, further comprising repeating the method following administration of drug therapy to the patient to detect drug resistance or effectiveness.

18. A method of detecting a pathogen present in a patient, the method comprising:
delivering an electromagnetic beam from the infrared-visible spectrum, through a substrate in contact with a sample derived from a patient biofluid to create an infrared sample spectrum representative of the biofluid, the sample spectrum having one or more spectral components, each component having a wavenumber and absorbance value,
analysing the absorbance value of at least one spectral components to detect the presence of DNA from a pathogen by:
providing a reference database of spectral models, each model having one or more database spectral components of a wavenumber and an absorbance value, wherein the database spectral components identify disease agents;
identifying one or more database spectral components of the reference database matching or corresponding to one or more sample spectral components; and
compiling a list of matched database components identified.

19. The method of claim 18, wherein analysing the absorbance value of at least one spectral component comprises analysing less than all of the absorbance values of the at least one spectral component.

20. The method according to claim 18 or 19, further comprises quantifying the pathogen by comparing absorption of the electromagnetic beam with one or more calibration models to quantify the number of pathogen cells present in the biofluid.

21. The method according to claim 18, further comprising selecting a spectral window to reduce the one or more database spectral components and to reduce the one or more sample spectral components used for identifying matches or correspondences.

22. The method according to claim 19, wherein the electromagnetic beam is an evanescent infrared beam which is delivered through an ATR substrate in contact with the sample.

23. The method according to claim 19, wherein the sample is generated by extracting material from a patient biofluid, by:
isolating a particulate sample from the biofluid;
suspending the particulate sample in a solvent; and
concentrating the particulate sample in the solvent to form concentrated sample.

24. The method according to claim 19, further comprising centrifuging the biofluid the presence of added particles.

25. The method according to claim 19, wherein the pathogen is associated with sepsis.

26. The method according to claim 19, further comprising:
identifying a molecular phenotype which is specific for a particular type of pathogen by comparison of absorption of the infrared beam by the test sample with a database or a spectral model for the molecular phenotype.

27. A computer readable storage medium for storing in non-transient form an application for executing a method of detecting a pathogen associated with sepsis in a sample derived from a patient biofluid, the method comprising:
recording an infrared spectrum representative of the sample;
comparing said spectrum to a reference database of spectral models to identify one or more spectral components of wavenumber and absorbance of the sample, wherein the spectral components identify pathogens; and
compiling a list of sample components identified corresponding to a respective spectral model of the database;
wherein the recording, comparing and compiling are performed without user input.

28. The computer readable storage medium according to claim 27, wherein the method further comprises comparing said spectrum to a reference database of calibration models to identify one or more spectral components of wavenumber and absorbance of the sample, wherein the spectral components quantify the number of pathogenic cells present in the sample, and wherein the comparing is performed without user input.

29. The system of claim 1, wherein the controller is further configured to quantify the spectral components and output a pathogen load value.

30. The system of claim 29, wherein the pathogen load value is a colony forming unit value.

31. The system of claim 1 or 2, wherein each reference spectral model was generated using a reference sample that were prepared using a filter.

32. The system of claim 31, wherein the reference sample were further prepared by suspending the reference sample in a solvent and concentrating the particulate sample in an amount of solvent to form a concentrated reference sample.
